(19) United States
(12) Patent Application Publication
(10) Pub. No.: US 2019/0010547 A1
(43) Pub. Date: Jan. 10, 2019

(54) MICRORNA-122 IN METABOLIC DISEASES

(71) Applicants: Medizinische Universität Innsbruck, Innsbruck (AT); King's College London, London (GB)

(72) Inventors: Peter Willeit, Innsbruck (AT); Manuel Mayr, London (GB); Johann Willeit, Innsbruck (AT); Stefan Kiechl, Zirl (AT)

(73) Assignees: Medizinische Universität Innsbruck, Innsbruck (AT); King's College London, London (GB)

(21) Appl. No.: 15/774,001

(22) PCT Filed: Nov. 4, 2016
(86) PCT No.: PCT/EP2016/076631

§ 371 Date: May 5, 2018

(30) Foreign Application Priority Data
Nov. 6, 2015 (EP) 15193448.6

(51) Int. Cl. C12Q 1/6883

(52) U.S. Cl. CPC C12Q 1/6883; C12Q 2600/178; C12Q 2600/118

(57) ABSTRACT

The present invention relates to a predictive diagnostic method for identifying whether a patient will develop metabolic syndrome and/or type-2 diabetes in the future. In particular, the invention relates to a method for identifying a subject which has a risk for developing metabolic syndrome and/or type-2 diabetes, wherein the method comprises (a) analyzing (i.e. determining/measuring/quantifying) in a sample obtained from a test subject the amount of miR-122; and (b) identifying (i.e. detecting) a subject, which has a risk for developing metabolic syndrome and/or type-2 diabetes, wherein an increased amount of miR-122 as compared to the amount of miR-122 of a healthy reference population indicates a risk for developing metabolic syndrome and/or type-2 diabetes. Another aspect of the invention relates to a method for monitoring the therapeutic success during the treatment of metabolic syndrome and/or type-2 diabetes, wherein the method comprises (a) analyzing (i.e. determining/measuring/quantifying) in a first sample obtained from a test subject the amount of miR-122, wherein said first sample was obtained before or under treatment of the test subject with medication for metabolic syndrome and/or type-2 diabetes; (b) analyzing (i.e. determining/measuring/quantifying) in a second sample of said test subject the amount of miR-122, wherein said second sample was obtained under or after treatment of the test subject with medication for metabolic syndrome and/or type-2 diabetes; and (c) predicting the therapeutic success (i.e. detecting, whether therapeutic success exists), wherein a decreased amount of miR-122 in the second sample as compared to the first sample indicates therapeutic success.

The present invention relates to a predictive diagnostic method for identifying whether a patient will develop metabolic syndrome and/or type-2 diabetes in the future. In particular, the invention relates to a method for identifying a subject which has a risk for developing metabolic syndrome and/or type-2 diabetes, wherein the method comprises (a) analyzing (i.e. determining/measuring/quantifying) in a sample obtained from a test subject the amount of miR-122; and (b) identifying (i.e. detecting) a subject, which has a risk for developing metabolic syndrome and/or type-2 diabetes, wherein an increased amount of miR-122 as compared to the amount of miR-122 of a healthy reference population indicates a risk for developing metabolic syndrome and/or type-2 diabetes. Another aspect of the invention relates to a method for monitoring the therapeutic success during the treatment of metabolic syndrome and/or type-2 diabetes, wherein the method comprises (a) analyzing (i.e. determining/measuring/quantifying) in a first sample obtained from a test subject the amount of miR-122, wherein said first sample was obtained before or under treatment of the test subject with medication for metabolic syndrome and/or type-2 diabetes; (b) analyzing (i.e. determining/measuring/quantifying) in a second sample of said test subject the amount of miR-122, wherein said second sample was obtained under or after treatment of the test subject with medication for metabolic syndrome and/or type-2 diabetes; and (c) predicting the therapeutic success (i.e. detecting, whether therapeutic success exists), wherein a decreased amount of miR-122 in the second sample as compared to the first sample indicates therapeutic success.

MicroRNA-122 (miR-122) accounts for up to 70% of the small non-coding RNAs in the liver and has been proposed to play a central role in lipid and glucose homeostasis, and in hepatic insulin resistance (Fernández-Hernando, Arterioscler Thromb Vasc Biol 2013, 33: 178-85; Shantikumar, Cardiovascular Research 2012, 93: 583-93). Evidence from mice (Esau, Cell Metab 2006, 3: 87-98; Krützfeldt, Nature 2005, 438: 685-9) and non-human primates (Elmén, Nature 2008, 452: 896-9; Lanford, Science 2010, 327: 198-201) showed that inhibition of miR-122 induces fatty acid oxidation and reduces lipid synthesis, and thereby leads to lower levels of total cholesterol. MiR-122 has been suggested to be one of several biomarkers that may be useful in the diagnoses for several diseases (WO 2011/110644 A1). Use of anti-miR-122 has been suggested for therapy of various diseases (WO 2009/109665 A1). In addition, it was shown that proanthocyanididis normalized liver mirR-122 levels in high-fat diet-induced obese rats (Baselga-Escudero, Nutrition research 2015, 35: 337-345). Furthermore, miR-122 is elevated in response to various liver diseases, including non-alcoholic fatty liver disease, non-alcoholic steatohepatitis, and viral hepatitis, probably reflecting tissue damage (Szabo, Nat Rev Gastroenterol Hepatol 2013, 10: 542-52; Bandiera, J Hepatol 2015, 62: 448-57).

While experimental evidence suggests that miR-122 may have adverse metabolic effects and contribute to the development of cardiometabolic diseases, the long-term relevance of circulating miR-122 in humans is largely unknown. Studies in human populations into the relationship of miR-122 with cardiometabolic traits are sparse and have critical limitations. Previous studies have focused on correlations with major lipid classes (Gao, Lipids Health Dis 2012, 11: 55), whereas a breakdown into lipid subspecies would add resolution and improve the interpretation of the regulation of lipid homeostasis by miR-122. Also, studies were cross-sectional or had short-term follow-up durations, whereas a long-term prospective study would enable to establish a temporal relationship between miR-122 and disease incidence, reduce the scope for biases, and allow more valid inferences to be made.

Metabolic syndrome is defined by a constellation of an interconnected physiological, biochemical, clinical, and metabolic factors that directly increases the risk of atherosclerotic cardiovascular disease and type-2 diabetes. The prevalence of metabolic syndrome and type-2 diabetes are about 25% (International Diabetes Federation) and about 9% (WHO 2014), respectively. The absolute number of diabetes patients worldwide has grown by a factor of five to almost 500 million over a period of 35 years. Biomarkers to estimate predisposition to metabolic syndrome or type-2 diabetes are very important, as an early regulation of diet and life style changes have considerable effect on the development and progress of these disease.

In addition, after manifestation of metabolic syndrome or type-2 diabetes, the well-being of the patient considerably depends on an appropriate therapy. In particular, early identification of whether a patient responds to a selected therapeutic approach increases the chance of recovery and prevents unnecessary suffering of the patient from side-effects of non-effective medicaments.

Thus, the technical problem underlying the present invention is the provision of means and methods for early diagnosis of metabolic syndrome and/or type-2 diabetes before onset of the disease(s) and at a time point where life-style interventions can delay or prevent outbreak and/or severity of the disease(s) and/or prevent complications; as well as for monitoring therapeutic success during the treatment of metabolic syndrome and/or type-2 diabetes.

The technical problem is solved by the provision of the embodiments as characterized in the claims.

Accordingly, the present invention relates to a diagnostic method for identifying predisposition to metabolic syndrome and/or type-2 diabetes in an asymptotic subject. Particularly the invention relates to a method for identifying a subject which has a risk for developing metabolic syndrome and/or type-2 diabetes before onset of the disease(s), wherein the method comprises:
(a) analyzing in a sample obtained from a test subject the amount of miR-122; and
(b) identifying a subject, which has a risk for developing metabolic syndrome and/or type-2 diabetes, wherein an increased amount of miR-122 as compared to the amount of miR-122 of a healthy reference population indicates a risk for developing metabolic syndrome and/or type-2 diabetes.

Thus, the above described diagnostic method can be used to identify a subject which has a risk for developing metabolic syndrome and/or type-2 diabetes before onset of the disease(s), i.e. before onset of clinical symptoms of the disease(s). For example, the above described diagnostic method can be used to identify a subject which has a risk for developing metabolic syndrome and/or type-2 diabetes at least 1 year, e.g. at least 2 years, at least 3 years, at least 4 years, or at least 5 years before onset of the disease(s). For example, the diagnostic method can be used to identify a subject which has a risk for developing metabolic syndrome and/or type-2 diabetes from 10 years before onset of the disease(s) until directly before (e.g. one month before) onset of the disease(s). Preferably, the diagnostic method is used to identify a subject which has a risk for developing metabolic syndrome and/or type-2 diabetes from 7.5 years before onset of the disease(s) until directly before (e.g. one month before) onset of the disease(s). More preferably, the diagnostic method is used to identify a subject which has a risk for developing metabolic syndrome and/or type-2 diabetes from 5 years before onset of the disease(s) until directly before (e.g. one month before) onset of the disease(s). “Onset of the disease(s)” means outbreak of the disease(s), i.e. clinical manifestation of the disease(s), particularly of metabolic syndrome and/or type-2 diabetes. Preferably, the amount of circulating miR-122 is analyzed in the herein provided diagnostic method. “Circulating miR-122” is the miR-122 that is transported by the blood, i.e. miR-122 present in blood, blood plasma or blood serum. Thus, metabolic syndrome and/or type-2 diabetes may break out within 10 years, preferably within 7.5 years, more preferably within 5 years after a risk for developing metabolic syndrome and/or type-2 diabetes has been identified by the herein provided diagnostic method.

The present invention solves the above identified technical problem since, as documented herein below and in the appended examples, it was surprisingly found that miR-122 is a biomarker for identifying (i.e. diagnosing) the risk for developing metabolic syndrome and type-2 diabetes long before onset of the disease(s). In the appended Examples miR-122 was measured in serum samples of 810 patients (in the year 1995) and in a reexamination 5 years later (in the year 2000) in serum and plasma samples of 695 patients. In addition, in this reexamination it was tested whether the patients have developed a metabolic syndrome and/or type-2 diabetes. The results convincingly show strong and stable increase of miR-122 in patients 5 years before onset of metabolic syndrome and/or type-2 diabetes. Thus, the appended Examples surprisingly demonstrate that miR-122 levels are associated with the long-term risk of developing metabolic syndrome and type-2 diabetes. Accordingly, the present invention relates to a diagnostic method for identifying an asymptotic subject which has an increased probability (i.e. an increased risk) for developing metabolic syndrome and/or type-2 diabetes.

In the prior art miR-122 has been mentioned in the context of several diseases including metabolic syndrome and type-2 diabetes (Rotllan, Cholesterol 2012: 1-8; Kaur, World Journal of Diabetes, 2011, 2: 158-163; and “Nutritional Intervention in Metabolic Syndrome”, edited by Isaias Dichi, Andrea Name Colado Simão, CRC press, 2016). However, it has never been suggested to use miR-122 as a biomarker for metabolic syndrome or type-2 diabetes before onset of the disease. However, detection of the risk for developing these diseases before manifestation of the disease has the advantage that the affected persons can change their life style (e.g. reducing their weight-to-hip ratio) in order to prevent outbreak or to decrease severity of the diseases.

MicroRNAs (miRNA, mir-) are emerging as important regulators and potential biomarkers in different disease. miRNA are small noncoding RNAs that function as posttranscriptional regulators of gene expression. Using miRNAs (such as miR-122) as a biomarker has several advantages. For example, miRNAs are present intra- and extracellularly, and are very stable in body fluids. Due to the extracellular stability, analysis of a miRNA can be used in the diagnosis of complex systemic diseases.

MiR-122 is one of the very few miRNAs that have organ specificity and can be measured in blood serum as well as blood plasma. Thus, miR-122 is a highly specific marker that can easily be detected in blood samples during routine physicals.

Thus, the invention relates to a method for identifying a risk for developing metabolic syndrome and/or type-2 diabetes, wherein the method comprises:
(a) analyzing (i.e. determining/quantifying/measuring) in a sample the amount of miR-122; and
(b) identifying (i.e. detecting/determining/evaluating) a risk for developing metabolic syndrome and/or type-2 diabetes, wherein an increased amount of miR-122 in said sample as compared to the amount of miR-122 of a healthy reference population indicates a risk for developing metabolic syndrome and/or type-2 diabetes.

In the inventive method, the risk for developing metabolic syndrome and/or type-2 diabetes is identified (i.e. diagnosed) in a test subject (preferably a human being). The sample, wherein the amount of miR-122 is analyzed (i.e. determined/quantified/measured) is obtained from said test subject.

Accordingly, the invention relates to a method for identifying a subject which has a risk for developing metabolic syndrome and/or type-2, wherein the method comprises:
(a) analyzing in a sample obtained from a test subject the amount of miR-122; and
(b) identifying a subject, which has a risk for developing metabolic syndrome and/or type-2 diabetes, wherein an increased amount of miR-122 as compared to the amount of miR-122 of a healthy reference population indicates a risk for developing metabolic syndrome and/or type-2 diabetes.

Preferably, the amount of circulating miR-122 is analyzed in the herein described diagnostic method.

As described above, according to the invention, miR-122, particularly circulating miR-122, can be used as a biomarker for identifying (i.e. diagnosing) the risk for developing metabolic syndrome and/or type-2 diabetes. Accordingly, the present invention provides a predictive biomarker. More specifically, the present invention provides miR-122 as a predictive biomarker for use in identifying a risk for developing a metabolic syndrome and/or type-2 diabetes, wherein an increased amount of miR-122 as compared to the amount of miR-122 of a healthy reference population indicates a risk for developing metabolic syndrome and/or type-2 diabetes. As described above, miR-122 has the advantage that it can be used to diagnose the risk for developing metabolic syndrome and/or type-2 diabetes before onset of the disease(s). Accordingly, the present invention relates to miR-122 as a predictive biomarker for use in identifying a risk for developing metabolic syndrome and/or type-2 diabetes before onset of the disease(s), wherein an increased amount of miR-122 as compared to the amount of miR-122 of a healthy reference population indicates a risk for developing metabolic syndrome and/or type-2 diabetes. The miR-122 that is used as a biomarker as described herein is preferably circulating miR-122.

It is very common that patients develop both diseases, metabolic syndrome and type-2 diabetes. Usually metabolic syndrome manifests first and type-2 diabetes later on in the same subject. However, sometimes patients with metabolic syndrome already fulfill the criteria of type-2 diabetes. Vice versa almost all patients with type-2 diabetes fulfill the criteria of metabolic syndrome. Thus, the herein provided diagnostic method or the herein provided biomarker can be used to identify a risk for developing either metabolic syndrome or type-2 diabetes, or for identifying a risk for developing both, metabolic syndrome and type-2 diabetes. For example, the herein provided diagnostic method or the herein provided biomarker can be used to identify a subject which has a risk for developing type-2 diabetes. Alternatively, the herein provided diagnostic method or the herein provided biomarker can be used for identifying a risk for developing metabolic syndrome.

With the herein provided diagnostic method, or by using the biomarker as described herein, the risk for developing metabolic syndrome and/or type-2 diabetes can be identified long before clinical manifestation of these diseases, for example from 10 years before clinical manifestation of metabolic syndrome and/or type-2 diabetes until manifestation of the disease. It is preferred that the risk for developing metabolic syndrome and/or type-2 diabetes is identified at least 1 year before clinical manifestation of the metabolic syndrome and/or the type-2 diabetes, respectively. For example, the herein provided diagnostic method or the herein provided biomarker can be used to identify a subject which has a risk for developing metabolic syndrome and/or type-2 diabetes at least 1 year, at least 2 years, at least 3 years, at least 4 years, or at least 5 years before onset of the disease(s). Accordingly, the diagnostic method can be used to identify a subject which has a risk for developing metabolic syndrome and/or type-2 diabetes from 10 years before onset of the disease(s) until directly before (e.g. one month before) onset of the disease(s). Preferably, the diagnostic method is used to identify a subject which has a risk for developing metabolic syndrome and/or type-2 diabetes from 7.5 years before onset of the disease(s) until directly before (e.g. one month before) onset of the disease(s). More preferably, the diagnostic method is used to identify a subject which has a risk for developing metabolic syndrome and/or type-2 diabetes from 5 years before onset of the disease(s) until directly before (e.g. one month before) onset of the disease(s). Thus, metabolic syndrome and/or type-2 diabetes may break out within 10 years, preferably within 7.5 years, more preferably within 5 years after a risk for developing metabolic syndrome and/or type-2 diabetes has been identified by the herein provided diagnostic method or by using the herein provided biomarker.

Usually, medication for metabolic syndrome or type-2 diabetes will not be initiated until diagnosis of the disease. Thus, when applying the herein provided diagnostic method or when applying the biomarker as described herein, the risk for developing a metabolic syndrome and/or type-2 diabetes may be identified in a test subject which did not receive medication for a metabolic syndrome and/or type-2 diabetes.

Manifestation of metabolic syndrome and/or type-2 diabetes can be performed according to standardized diagnostic criteria. For example, metabolic syndrome may be diagnosed if three out of the five following characteristics are present (Alberti, Circulation 2009, 120: 1640-5): (i) waist circumference in men ≥102 cm and women ≥88 cm; (ii) fasting triglycerides ≥150 mg/dl or on drug treatment for elevated triglycerides (e.g. on fibrates and nicotinic acids); (iii) HDL cholesterol in men <40 and women <50 mg/dl or on drug treatment for reduced HDL cholesterol (e.g. on fibrates and nicotinic acids); (iv) blood pressure ≥130/≥85 mmHg or antihypertensive drug treatment in a patient with a history of hypertension; and (v) fasting glucose ≥100 mg/dl or on drug treatment for elevated glucose. Type-2 diabetes may be diagnosed according to 1997 American Diabetes Association criteria, recent updates thereof, WHO criteria, or other.

The risk for developing metabolic syndrome or type-2 diabetes that can be detected by the herein provided means and methods can be quantified by the provision of specific risk ratios. These risk ratios are a measure of the strength of association between the miR-122 level and development of disease. In particular, the appended Examples show that an increased level of miR-122 indicates a risk for developing metabolic syndrome with a risk ratio of about 1.8-4.6. More specifically, the appended Examples show that 1.8-4.6 is the 95% confidence interval of the risk ratio for metabolic syndrome, comparing the risk in the top third of miR-122 levels (i.e., >66th percentile) vs. the risk in the bottom third of miR-122 levels (i.e., <33th percentile). Thus, for metabolic syndrome the range of 1.8-4.6 is the 95% confidence interval. This is the range in which—with 95% confidence—the “true” association between an increase miR-122 level and development of metabolic syndrome lies. Thus, the invention relates to the herein provided diagnostic method or to the biomarker as provided herein, wherein the method or the biomarker is for identifying a subject which has a risk for developing metabolic syndrome, and wherein the risk ratio is 1.8-4.6.

The appended Examples also show that an increased level of miR-122 indicates a risk for developing type-2 diabetes with a risk ratio of 1.3-6.4. The range 1.3-6.4 is the 95% confidence interval of the risk ratio for type-2 diabetes, comparing the risk in the top third of miR-122 levels (i.e., >66th percentile) vs. the risk in the bottom third of miR-122 levels (i.e., <33th percentile). Thus, the present invention relates to the herein provided diagnostic method or the herein provided biomarker, wherein the method or the use is for identifying a subject which has a risk for developing type-2 diabetes, and wherein the risk ratio is 1.3-6.4.

In context of the present invention, the risk ratios are preferable age- and sex-adjusted, meaning that they have been corrected for any influences age or sex may have (i.e. the association between an increased miR-122 level and the development of metabolic syndrome and/or type-2 diabetes is “independent” of age and sex).

The present invention relates to the herein provided diagnostic method or the herein provided biomarker, wherein miR-122 is a polynucleotide selected from the following polynucleotides
(i) a polynucleotide comprising or consisting of the nucleotide sequence of SEQ ID NO: 1;
(ii) a polynucleotide which is at least 95% identical to the nucleotide sequence of SEQ ID NO: 1 and being functional, wherein the function comprises the activity to repress translation of the target genes of miR-122 (e.g. the mouse genes Acaca, Acly, Fasn, Scd1, Srebf1); and
(iii) a polynucleotide according to (ii), which comprises the nucleotide sequence of SEQ ID NO: 2.

Item (ii), above, refers to a polynucleotide which is at least 95%, preferably at least 96%, more preferably at least 97%, even more preferably at least 98%, even more preferably at least 99% identical to the nucleotide sequence of SEQ ID NO: 1 and being functional, wherein the function comprises the activity to repress translation of the target genes of miR-122 (e.g. the mouse genes Acaca, Acly, Fasn, Scd1, Srebf1). Preferably, in context of the present invention miR-122 is the polynucleotide as described in (i) or
(iii) above. More preferably, miR-122 is the polynucleotide as described in (i) above (i.e. a polynucleotide comprising or consisting of the nucleotide sequence of SEQ ID NO: 1). Most preferably, miR-122 is a polynucleotide consisting of the nucleotide sequence of SEQ ID NO: 1.

miR-122 exerts its biological activity by binding to the 3′-untranslated region (3′-UTR) of its target mRNAs and thereby repressing their translation or by inducing mRNA degradation (Huntzinger Nat Rev Genet 2011; 12(2):99-110). In mice, inhibition of miR-122 leads to an upregulation of target genes via unknown mechanisms, thus regulating lipid metabolism in the liver. The target genes of miR-122 include the genes Acaca, Acly, Fasn, Scd1, Srebf1 (mouse gene names) (Tsai, J Clin Invest 2012; 122: 2884-97; Esau, Cell Metab 2006; 3: 87-98; Elmen, Nucleic Acids Res 2008; 36: 1153-62). Expression analysis of these genes (i.e. of the mouse genes Acaca, Acly, Fasn, Scd1, Srebf1) could be used to assay miR-122 activity. MiR-122 also increases the replication of the hepatitis C virus (HCV) by binding the 5′-UTR of HCV RNA (Roberts, Nucleic Acids Res 2011; 39: 7716-29). Inhibition of miR-122 reduces HCV RNA levels (Janssen, N Engl J Med 2013; 368: 1685-94).

In the herein provided diagnostic method or by using the herein provided biomarker for identifying a risk for developing metabolic syndrome and/or type-2 diabetes, the healthy reference population preferably consists of at least 20 healthy subjects (e.g. 20-1000 healthy human beings).

If the herein provided diagnostic method or the herein provided biomarker is for identifying the risk for developing metabolic syndrome, the healthy reference population (which preferably consists of healthy human beings) does not have metabolic syndrome and does not have a risk for developing metabolic syndrome. Thus, one aspect of the present invention relates to the diagnostic method provided herein, or the biomarker provided herein, wherein the method or the biomarker is for identifying a subject which has a risk for developing metabolic syndrome, and wherein said healthy reference population does not have a metabolic syndrome and fulfills at least one of the criteria (i) and (ii), below:
(i) does not have any of the features selected from
   (a) waist circumference of men that is ≥102 cm, or of women that is ≥88 cm;
   (b) fasting triglycerides ≥150 mg/dl, or being on drug treatment for elevated triglycerides (e.g. on drug treatment with fibrates and/or nicotinic acids);
   (c) HDL cholesterol in men that is <40, and in women that is <50 mg/dl or being on drug treatment for reduced HDL cholesterol (e.g. on drug treatment with fibrates and/or nicotinic acids);
   (d) blood pressure ≥130/85 mmHg, or being on antihypertensive drug treatment and having a history of hypertension; and
   (e) fasting glucose ≥100 mg/dl, or being on drug treatment for elevated glucose;
(ii) does have a miR-122 level beneath the 33^{th} percentile of population normative values.

A miR-122 level beneath the 33^{th} percentile of population normative values can be determined by measuring the amount of miR-122 (i.e. the miR-122 level) in a population to identify the miR-122 distribution. If the miR-122 level distribution is brought in ascending order, 33% of values lie below this value and the remainder (i.e. 66%) above it. Said population may be a population of at least 100 persons, e.g. a random population of 100-1000 persons.

In context of the present invention it has been found that persons with no risk for developing metabolic syndrome or type-2 diabetes have a median miR-122 value of 0.65 (interquartile range: 0.34-1.12).

If the herein provided diagnostic method or the herein provided biomarker is for identifying the risk for developing type-2 diabetes, the healthy reference population (which preferably consists of healthy human beings) does not have type-2 diabetes and does not have a risk for developing type-2 diabetes. Thus, one aspect of the present invention relates to the diagnostic method as provided herein, or the biomarker as provided herein, wherein the method or the biomarker is for identifying a subject which has a risk for developing type-2 diabetes, and wherein said healthy reference population does not have type-2 diabetes and does have a miR-122 level beneath the 33^{th} percentile of population normative values. For example, in the appended Examples a miR-122 value of <0.500 has been shown to be beneath the 33^{th} percentile of population normative values. As mentioned above, persons with no risk for developing metabolic syndrome or type-2 diabetes have a median miR-122 value of 0.65 (interquartile range: 0.34-1.12). In addition or alternatively, said healthy reference population may not have a fasting glucose of 126 mg/d L.

In the appended Examples, the amount of miR-122 has been determined as means and interquartile ranges. In particular, persons with a risk for developing metabolic syndrome have been shown to have a median miR-122 value of 1.05 (interquartile range: 0.65-2.93). Similarly, persons with a risk for developing type-2 diabetes have a median miR-122 value of 0.94 (interquartile range: 0.55-1.66). In contrast, persons with no risk for either of the two diseases have a median miR-122 value of 0.65 (interquartile range: 0.34-1.12). These numbers are unitless as they were generated with quantitative polymerase chain reaction (allowing relative quantification only). In particular, these values reflect the miR-122 amount relative to the amount of a standard (particularly relative to the amount of U6 and Cel-miR-39). Thus, a median miR-122 value of 1 would mean that the amount of miR-122 and the amount of the standard is identical. Accordingly, in context of the present invention for analyzing the amount of miR-122 U6 and exogenous C. elegans spike-in control (Cel-miR-39) may be used for normalisation purposes.

However, also absolute quantification of the amount (i.e. level) of miR-122 may be performed in context of the invention. For example, absolute quantification would be possible with the use of standard curves (involving spiking-in of reference microRNA samples with known concentrations é generation of standard curves é inference of absolute concentrations in the patient samples).

The term “interquartile range” refers to the range between the 25^{th} percentile and the 75^{th} percentile. The “interquartile range” can be determined by measuring the amount of miR-122 (i.e. the miR-122 level) in a population to identify the miR-122 distribution. If the miR-122 level distribution is brought in ascending order, 25% of values lie below the interquartile range and 25% lie above it. Said population may be a population of at least 100 persons, e.g. a random population of 100-1000 persons.

In the context of the present invention it has been found that in persons that have a manifested metabolic syndrome the amount of miR-122 is increased by 160% (i.e. to a value of 260%) as compared to the healthy reference population. Similarly, in persons that have manifested type-2 diabetes the amount of miR-122 is increased by 214% (i.e. to a value of 314%) as compared to the healthy reference population. Surprisingly, already 5 years prior to manifestation of these diseases, the affected persons showed an increased level of miR-122. In particular, 5 years before manifestation of the metabolic syndrome the amount of miR-122 was already increased by 104% (i.e. to a value of 204%) as compared to the healthy reference population. Similarly, 5 years before manifestation of type-2 diabetes, the miR-122 level was already increased by 49% (i.e. to a value of 149%) as compared to the healthy reference population.

Thus, in one aspect of the herein provided diagnostic method or the herein provided biomarker an amount of miR-122 of the test subject, which is at least 110% (preferably at least 120%, more preferably at least 125%, even more preferably at least 130%, even more preferably at least 135%) of the amount of miR-122 of the healthy reference population, indicates the risk for developing metabolic syndrome and/or type-2 diabetes. For example, if the herein provided diagnostic method or the herein provided biomarker is for identifying a risk for developing metabolic syndrome, an amount of miR-122 of the test subject, which is at least 110% (preferably at least 120%, more preferably at least 130%, even more preferably at least 140%, even more preferably at least 150%, even more preferably at least 160%, even more preferably at least 170%, even more preferably at least 180%, or even more preferably at least 190%) of the amount of miR-122 of the healthy reference population, indicates the risk for developing metabolic syndrome. In addition or alternatively, if the herein provided diagnostic method or the herein provided biomarker is for identifying a risk for developing type-2 diabetes, an amount of miR-122 of the test subject, which is at least 110% (preferably at least 120%, more preferably at least 125%, even more preferably at least 130%, even more preferably at least 135%, or even more preferably at least 140%) of the amount of miR-122 of the healthy reference population, indicates the risk for developing type-2 diabetes.

If the risk for developing metabolic syndrome and/or type-2 diabetes is predicted by using the herein provided diagnostic method or by using the biomarker as provided herein, it may also be analyzed (i.e. determined/measured) whether the test subject has conventional risk factors for metabolic syndrome and/or type-2 diabetes. Thus, one aspect of the present invention relates to the herein provided diagnostic method or the herein provided biomarker, further comprising identifying whether said test subject has at least one of the risk factors selected from overweight (BMI≥25), obesity (BMI≥30), central obesity (waist circumference of men that is ≥102 cm, or of women that is ≥88 cm), hypertension (blood pressure ≥140/90), low HDL cholesterol (HDL cholesterol in men that is <40, and in women that is <50 mg/dl), and high triglyceride levels (≥150 mg/dL). These features are risk factors for both diseases, metabolic syndrome and type-2 diabetes.

The herein provided diagnostic method and miR-122 as a biomarker as described herein have the advantage that the risk for developing metabolic syndrome and/or type-2 diabetes can be detected very early, i.e. long before onset of the disease(s). Thus, the patient that has been identified as having a risk for developing metabolic syndrome and/or type-2 diabetes has the possibility to delay or prevent outbreak of the disease, e.g. by life-style interventions or a medication for metabolic syndrome and/or type-2 diabetes. Thus, a further aspect of the present invention relates to the herein provided diagnostic method or the herein provided biomarker, wherein a medication for a metabolic syndrome and/or type-2 diabetes is to be administered to the test subject which has been identified as having a risk for developing metabolic syndrome and/or type-2 diabetes; and/or wherein life-style interventions are recommended to the test subject which has been identified as having a risk for developing metabolic syndrome and/or type-2. The test subject which has been identified as having a risk for developing metabolic syndrome and/or type-2 diabetes may also be subjected to follow ups in order to monitor disease status of the subject at risk.

The medication may comprise or consist of metabolic syndrome or type-2 diabetes medicaments that are in accordance with standard guidelines. For example, a medication for type-2 diabetes may include or consist of metformin treatment. A medication for metabolic syndrome may include or consist of statin (e.g. atorvastatin) treatment. Thus, a test subject (i.e. patient) that has been identified as having a risk for developing metabolic syndrome may be treated with statin (e.g. atorvastatin). Said medication for metabolic syndrome may also be antagomiR-122 treatment.

Thus, one aspect of the invention relates to a method of treating a subject in need of such a treatment with medication for metabolic syndrome and/or type-2 diabetes before the outbreak of the disease(s), wherein said subject has been identified as having a risk for developing metabolic syndrome and/or type-2 diabetes, respectively, and wherein said treatment method comprises administering medication for metabolic syndrome and/or type-2 diabetes to the subject. Said medication may be a miR-122 inhibitor such as an antisense oligonucleotide against miR-122. For example, said miR-122 inhibitor may be antagomiR-122. It is envisaged that said subject in need of such a treatment has been identified as having a risk for developing metabolic syndrome and/or type-2 diabetes by using the diagnostic method or the biomarker of the present invention.

The life-style interventions (that are recommended to the subject at risk) may be, for example increased physical activity and/or reduction of the amount of calories consumed per day and/or other standard dietary recommendations. These interventions may result in a decreased waist-to-hip ratio counteracting the development and severity of metabolic syndrome and type-2 diabetes.

In the herein provided diagnostic method or when using the biomarker as described herein, several different samples can be used for measuring the amount (i.e. the level) of miR-122. In one aspect of the invention, said sample is blood, blood plasma, blood serum, urine or a liver tissue sample. Preferably, the sample is blood serum or blood plasma. Most preferably the sample is blood serum. In context of the invention, said amount of miR-122 may be analyzed by quantitative PCR (polymerase chain reaction).

More specifically, the amount (i.e. the level) of miR-122 may be measured as previously described (Willeit, Circ Res 2013, 112: 595-600; Zampetaki, Circ Res 2010, 107: 810-7). Briefly, miRNAs may be extracted, e.g. by using the miRNeasy kit (Qiagen, Hilden, Germany). For plasma or serum samples a fixed volume of 3 μl of the 25 μl RNA eluate may be used as input for reverse transcription (RT) reactions. For RNA from cells or tissue, 100 ng input material may be used for RT. MiRNAs may be reversely transcribed using Megaplex Primer Pools (Human Pools A version 2.1 or Rodent Pool A, Life Technologies, Darmstadt, Germany) and products may be further amplified using Megaplex PreAmp Primers (Primers A v2.1). Both RT and PreAmp products may be stored at −20° C. Taqman miRNA assays may be used to assess the expression of miR-122. Diluted pre-amplification product (0.5 μl) or RT product (corresponding to 0.45 ng input) may be combined with 0.25 μl Taqman microRNA assay (20×) (Life Technologies) and 2.5 μl Taqman Universal PCR Master Mix No AmpErase UNG (2×) to a final volume of 5 μl. A qRT-PCR may be performed on an Applied Biosystems 7900HT thermocycler at 95° C. for 10 min, followed by 40 cycles of 95° C. for 15 s and 60° C. for 1 min. All samples may be run in duplicates. Relative quantification may be performed using the software SDS2.2 (Life Technologies). U6 and exogenous C. elegans spike-in control (Cel-miR-39) may be used for normalisation purposes.

However, the amount of miR-122 may also be analyzed (i.e. determined) by using a binding molecule, which specifically binds to miR-122. The binding molecule which specifically binds to miR-122 may be an oligonucleotide (i.e. a probe or primer). The production of such oligonucleotides is commonly known in the art. The binding molecule, which is in context of the invention used for identifying a subject which has a risk for developing metabolic syndrome and/or type-2 diabetes can be part of a kit, which may be used to diagnose the risk for developing metabolic syndrome and/or type-2 diabetes before onset of the disease(s). Thus, the invention relates to the use of a kit for identifying a subject which has a risk for developing metabolic syndrome and/or type-2 diabetes before onset of the disease(s), wherein the kit comprises the binding molecule as defined herein above. The herein provided inventive diagnostic method may be realized by using this kit. Advantageously, the kit of the present invention further comprises optionally (a) reaction buffer(s), storage solutions, wash solutions and/or remaining reagents or materials required for the conduction of the assays as described herein. Furthermore, parts of the kit of the invention can be packaged individually in vials or bottles or in combination in containers or multicontainer units. These vials/bottles/containers or multicontainers may, in addition to the binding molecule described herein, comprise preservatives or buffers for storage. In addition, the kit may contain instructions for use. The manufacture of the kit of the present invention follows preferably standard procedures which are known to the person skilled in the art. As mentioned above, the kit provided herein is useful for identifying a subject, which has a risk for developing metabolic syndrome and/or type-2 diabetes before onset of the disease(s).

The appended Examples show that established medications for metabolic syndrome (particularly statin treatment) reduce circulating miR-122 levels. A similar statin response was also observed in mice and cultured hepatocytes. Thus, the appended Examples demonstrate that miR-122 is a molecular marker that indicates successful treatment of diseases that go along with an increased miR-122 level. Accordingly, measuring the level of miR-122 can be used to identify whether medications for metabolic syndrome and/or type-2 diabetes lead to therapeutic success. Thus, the present invention relates to a method for monitoring the therapeutic success during the treatment of metabolic syndrome and/or type-2 diabetes, wherein the method comprises:

(a) analyzing in a first sample obtained from a test subject the amount of miR-122, wherein said first sample was obtained before or under treatment of the test subject with medication for metabolic syndrome and/or type-2 diabetes;
(b) analyzing in a second sample of said test subject the amount of miR-122, wherein said second sample was obtained under or after treatment of the test subject with medication for metabolic syndrome and/or type-2 diabetes; and
(c) predicting the therapeutic success, wherein a decreased amount of miR-122 in the second sample as compared to the first sample indicates therapeutic success.

Of course, in the above described monitoring method, the second sample was obtained after the first sample.

Also encompassed by the present invention is a monitoring method, wherein the amount of miR-122 of the test subject is compared to reference data. In particular, one aspect of the invention relates to a method for monitoring the therapeutic success during the treatment of metabolic syndrome and/or type-2 diabetes, wherein the method comprises:
(a) analyzing in a sample obtained from a test subject, which received or receives medication for metabolic syndrome and/or type-2 diabetes, the amount of miR-122;
(b) comparing said amount with reference data corresponding to the amount of miR-122 of a reference population; and
(c) predicting the therapeutic success based on the comparison step (b). If the reference population consists of healthy subjects (i.e. persons that do not have metabolic syndrome or type-2 diabetes), then an amount of miR-122 of the sample that is identical or similar to the reference data indicates therapeutic success. Similarly, if the reference population consists of diseased subjects (i.e. persons that have metabolic syndrome and/or type-2 diabetes) but which received medication for the disease, then an amount of miR-122 of the sample that is identical or similar to the reference data indicates therapeutic success. If the reference population consists of diseased subjects (i.e. persons that have metabolic syndrome and/or type-2 diabetes), then an amount of miR-122 of the sample that is below the reference data indicates therapeutic success.

In the above described monitoring method, “identical or similar amount of miR-122” means that the amount of miR-122 in the sample (e.g. in the blood plasma or blood serum sample) of the test subject is 60-140%, preferably 70-130%, more preferably 80-120%, and even more preferably 90-110% of amount of miR-122 of the reference population.

The above described monitoring methods may be used to monitor treatment success in the treatment of both (metabolic syndrome and type-2 diabetes) together. The above described monitoring methods may also be used to monitor treatment success in the treatment of either one of these disease, i.e. in the treatment of either metabolic syndrome or type-2 diabetes. Thus, one aspect of the present invention relates to a method for monitoring the therapeutic success during the treatment of metabolic syndrome, wherein the method comprises:
(a) analyzing in a first sample obtained from a test subject the amount of miR-122, wherein said first sample was obtained before or under treatment of the test subject with medication for metabolic syndrome;
(b) analyzing in a second sample of said test subject the amount of miR-122, wherein said second sample was obtained under or after treatment of the test subject with medication for metabolic syndrome; and
(c) predicting the therapeutic success, wherein a decreased amount of miR-122 in the second sample as compared to the first sample indicates therapeutic success.

It is preferred that the herein provided monitoring method is used for monitoring therapeutic success during the treatment of type-2 diabetes. Thus, another aspect of the present invention relates to a method for monitoring the therapeutic success during the treatment of type-2 diabetes, wherein the method comprises:
(a) analyzing in a first sample obtained from a test subject the amount of miR-122, wherein said first sample was obtained before or under treatment of the test subject with medication for type-2 diabetes;
(b) analyzing in a second sample of said test subject the amount of miR-122, wherein said second sample was obtained under or after treatment of the test subject with medication for type-2 diabetes; and
(c) predicting the therapeutic success, wherein a decreased amount of miR-122 in the second sample as compared to the first sample indicates therapeutic success.

The sample in the above described monitoring methods may by a blood serum or blood plasma sample, preferably a blood serum sample.

The monitoring methods as provided herein may also comprise analyzing the amount (i.e. the level) of further markers, wherein an altered (i.e. reduced or increased) amount of said markers further indicates therapeutic success in the treatment of metabolic syndrome and/or type-2 diabetes. Further markers that may be used in the herein provided monitoring methods are, e.g., fasting glucose and/or HbA_{1c}. In particular, in step (c) a decreased amount of fasting glucose and/or HbA_{1c} in the second sample as compared to the first sample indicates therapeutic success.

The definitions (particularly regarding the analysis of the amount of miR-122) disclosed herein in connection with the diagnostic method of the present invention (i.e. the method for identifying a subject which has a risk for developing metabolic syndrome and/or type-2 diabetes) apply, mutatis mutandis, to the monitoring method described above.

In the appended Examples, the amount of circulating miR-122 has been analyzed. Thus, in context of the herein provided diagnostic method, the herein provided biomarker or the herein provided monitoring method, said miR-122 is preferably circulating miR-122.

Thus, the present invention relates to the following items:
1. Method for identifying a subject which has a risk for developing metabolic syndrome and/or type-2 diabetes before onset of the disease(s), wherein the method comprises:
   (a) analyzing in a sample obtained from a test subject the amount of miR-122; and
   (b) identifying a subject, which has a risk for developing metabolic syndrome and/or type-2 diabetes, wherein an increased amount of miR-122 as compared to the amount of miR-122 of a healthy reference population indicates a risk for developing metabolic syndrome and/or type-2 diabetes.
2. MiR-122 as a predictive biomarker for use in identifying a risk for developing metabolic syndrome and/or type-2 diabetes before onset of the disease(s), wherein an increased amount of miR-122 as compared to the amount of miR-122 of a healthy reference population indicates a risk for developing metabolic syndrome and/or type-2 diabetes.
3. Method of item 1 or biomarker for the use according to item 2, wherein the risk for developing a metabolic syndrome and/or type-2 diabetes is identified at least 1 year before clinical manifestation of the metabolic syndrome and/or the type-2 diabetes, respectively.
4. Method of any one of items 1-3 or biomarker for the use according to item 2 or 3, wherein the method or the biomarker is for identifying a subject which has a risk for developing metabolic syndrome, and wherein the risk ratio is 1.8-4.6.
5. Method of any one of items 1-3 or biomarker for the use according to item 2 or 3, wherein the method or the biomarker is for identifying a subject which has a risk for developing type-2 diabetes, and wherein the risk ratio is 1.3-6.4.
6. Method of any one of items 1-5 or biomarker for the use according to any one of items 2-5, wherein miR-122 is a polynucleotide selected from the following polynucleotides
   (i) a polynucleotide comprising or consisting of the nucleotide sequence of SEQ ID NO: 1;
   (ii) a polynucleotide which is at least 95% identical to the nucleotide sequence of SEQ ID NO: 1 and being functional, wherein the function comprises the activity to repress translation of the target genes of miR-122; and
   (iii) a polynucleotide according to (ii), which comprises the nucleotide sequence of SEQ ID NO: 2.
7. Method of any one of items 1-4 and 6 or biomarker for the use according to any one of items 2-4 and 6, wherein the method or the biomarker is for identifying a subject which has a risk for developing metabolic syndrome, and wherein said healthy reference population does not have metabolic syndrome and fulfills at least one of the criteria (i) and (ii), below:
   (i) does not have any of the features selected from
      (a) waist circumference of men that is ≥102 cm, or of women that is ≥88 cm;
      (b) fasting triglycerides ≥150 mg/dl, or being on drug treatment for elevated triglycerides;
      (c) HDL cholesterol in men that is <40, and in women that is <50 mg/dl or being on drug treatment for reduced HDL cholesterol;
      (d) blood pressure ≥130/≥85 mmHg, or being on antihypertensive drug treatment and having a history of hypertension; and
      (e) fasting glucose ≥100 mg/dl, or being on drug treatment for elevated glucose; or
   (ii) does have a miR-122 level beneath the 33th percentile of population normative values.
8. Method of any one of items 1-3, 5 and 6 or biomarker for the use according to any one of items 2, 3, 5 and 6, wherein the method or the biomarker is for identifying a subject which has a risk for developing type-2 diabetes, and wherein said healthy reference population does not have type-2 diabetes and does have a miR-122 level beneath the 33th percentile of population normative values.
9. Method of any one of items 1-8 or biomarker for the use according to any one of items 2-8, wherein an amount of miR-122 of the test subject, which is at least 110% of the amount of miR-122 of the healthy reference population, indicates the risk for developing metabolic syndrome and/or type-2 diabetes.
10. Method of any one of items 1-9 or biomarker for the use according to any one of items 1-10, further comprising identifying whether said test subject has at least one of the risk factors selected from overweight, obesity, central obesity, hypertension, low HDL cholesterol or high triglyceride levels.
11. Method of any one of items 1-10 or biomarker for the use according to any one of items 2-10, wherein a medication for a metabolic syndrome and/or type-2 diabetes is to be administered to the test subject which has been identified as having a risk for developing metabolic syndrome and/or type-2 diabetes; and/or
   wherein life-style interventions are recommended to the test subject which has been identified as having a risk for developing metabolic syndrome and/or type-2.
12. Method of any one of items 1-11, wherein said sample is blood, blood plasma, blood serum, urine or a liver tissue sample.
13. Method of any one of items 1-12 or biomarker for the use according to any one of items 2-12, wherein said amount of miR-122 is analyzed by quantitative PCR.
14. Method for monitoring the therapeutic success during the treatment of metabolic syndrome and/or type-2 diabetes, wherein the method comprises:
   (a) analyzing in a first sample obtained from a test subject the amount of miR-122, wherein said first sample was obtained before or under treatment of the test subject with medication for metabolic syndrome and/or type-2 diabetes;
   (b) analyzing in a second sample of said test subject the amount of miR-122, wherein said second sample was obtained under or after treatment of the test subject with medication for metabolic syndrome and/or type-2 diabetes; and
   (c) predicting the therapeutic success, wherein a decreased amount of miR-122 in the second sample as compared to the first sample indicates therapeutic success.
15. Monitoring method of item 14, wherein the method is for monitoring the therapeutic success during the treatment of type-2 diabetes.
16. Monitoring method of item 14 or 15, wherein said sample is a blood serum or blood plasma sample.
17. Method of any one of items 1-13, biomarker for the use according to any one of items 2-13 or monitoring method of any one of items 14-16, wherein said miR-122 is circulating miR-122.

Herein the term “analyzing” means “determining”, “quantifying” or “measuring”. For example, herein the term “analyzing in a sample the amount of miR-122” means “determining, quantifying or measuring in a sample the amount of miR-122”. The amount of miR-122 can, e.g., be analyzed (i.e. quantified) by using quantitative PCR.

For the purposes of the present invention the “subject” (or “patient”) is most preferably a human being. However, the herein provided diagnostic method, biomarker or monitoring methods may also be applied for veterinary use. Accordingly, said subject may be an animal such as a mouse, rat, hamster, rabbit, guinea pig, ferret, cat, dog, chicken, sheep, bovine species, horse, camel, or primate. Preferably, the subject is a mammal. More preferably the subject is human. In addition, the test subject is preferably at moderately elevated CVD risk. Moderately elevated CVD risk means that the test subject has at least one of the risk factors selected from overweight (BMI≥25), obesity (BMI≥30), central obesity (waist circumference of men that is ≥102 cm, or of women that is ≥88 cm), hypertension (blood pressure≥140/90), low HDL cholesterol (HDL cholesterol in men that is <40, and in women that is <50 mg/dl), and high triglyceride levels (≥150 mg/dL). The experiments outlined in the appended Examples have been carried out by using a Caucasian population. Therefore, the “test subject” as well as the “reference population” may be Caucasian.

A “micro RNA”, (also called “microRNA”, “mir-”, “miR-” or “miRNA”) is a small noncoding RNA molecule (containing about 22 nucleotides) found in plants, animals, and some viruses, which functions in RNA silencing and post-transcriptional regulation of gene expression. MiRNAs function via base-pairing with complementary sequences within mRNA molecules. As a result, these mRNA molecules are silenced by one or more of the following processes: 1) cleavage of the mRNA strand into two pieces, 2) destabilization of the mRNA through shortening of its poly(A) tail, and 3) less efficient translation of the mRNA into proteins by ribosomes. MiRNAs are well conserved in both plants and animals, and are thought to be a vital and evolutionarily ancient component of genetic regulation. Herein the term “miR-” (particularly miR-122) refers to the mature miRNA. For example, miR-122 refers to a mature miRNA sequence derived from pre-miR-122. The sequence of miR-122 is shown herein as SEQ ID NO: 1.

The disease “metabolic syndrome” is also called “metabolic syndrome X”, “cardiometabolic syndrome”, “syndrome X”, “insulin resistance syndrome”, “Reaven's syndrome”, and “CHAOS”. Metabolic syndrome is a disease that is characterized by clustering of at least three of five of the following features: abdominal obesity, elevated blood pressure, dyslipidemia (high serum triglycerides, and low high-density lipoprotein (HDL) levels), elevated fasting plasma glucose, and insulin resistance. According to the present invention, the term “metabolic syndrome” also refers to all the other standard definitions of the disease “metabolic syndrome”, such as the definitions provided by the World Health Organization (WHO) or International Diabetes Federation (IDF).

Diabetes mellitus, commonly referred to as “diabetes”, is a group of metabolic diseases in which there are high blood sugar levels over a prolonged period. Symptoms of high blood sugar include frequent urination, increased thirst, and increased hunger. If left untreated, diabetes can cause many complications, such as diabetic ketoacidosis and nonketotic hyperosmolar coma. Serious long-term complications include cardiovascular disease, stroke, chronic kidney failure, foot ulcers, and damage to the eyes. Diabetes is due to either the pancreas not producing enough insulin or the cells of the body not responding properly to the insulin produced. There are three main types of diabetes mellitus, “type-1 diabetes”, “type-2 diabetes” and “gestational diabetes”. Type-2 diabetes (also called “type-2 diabetes mellitus”) begins with insulin resistance, a condition in which cells fail to respond to insulin properly. As the disease progresses a lack of insulin may also develop. Sometimes “type-2 diabetes” is also called “non insulin-dependent diabetes mellitus” (NIDDM) or “adult-onset diabetes”. The current standard definition of type-2 diabetes is a fasting glucose ≥126 mg/dL. However, according to the present invention, the term “type-2 diabetes” also refers to all the other standard definitions of the disease “type-2 diabetes”. The primary cause is excessive body weight and not enough exercise. As of 2014, an estimated 387 million people have diabetes worldwide, with type-2 diabetes making up about 90% of the cases.

In context of the present invention “risk ratios” are determined. Risk factors and risk ratios are commonly know in the art and can be ascertained by validated standard procedures as previously described (Willeit, Arterioscler Thromb Vasc Biol 2010, 30: 1649-56; Kiechl, N Engl J Med 2002, 347: 185-92).

The term “risk ratio” (also called “relative risk” or “RR”) is commonly known in the art and refers to the ratio of the probability of an event occurring (e.g. developing a disease) in an exposed group to the probability of the event occurring in a comparison, non-exposed group. For example, the risk ratio for developing metabolic syndrome or type-2 diabetes can be calculated by using the following formula:

$$RR = \frac{a / ( a + b )}{c / ( c + d )}$$

wherein

“a” is the probability of the persons with an increased miR-122 level to develop metabolic syndrome/type-2 diabetes; and “a+b=100”;

“c” is the probability of persons with a normal non-increased miR-122 to develop metabolic syndrome/type-2 diabetes; and “c+d=100”.

In context of the present invention, the risk ratio for metabolic syndrome may be calculated using logistic regression; the risk ratio for diabetes may be calculated using Cox regression. More specifically, miR-122 values may be log-transformed for analysis. Cross-sectional associations of miR-122 levels with other participant characteristics may be quantified using spearman correlation coefficients and linear regression models adjusted for age and sex. The prospective analysis uses Cox proportional hazard regression with updated covariates for type-2 diabetes and pooled logistic regression (D'Agostino, Stat Med 1990, 9: 1501-15) for metabolic syndrome. Hazard ratios and odds ratios may be assumed to represent the same measure of relative risk and are collectively described as risk ratios. Participants with prevalent disease may be excluded from the respective analyses. Models are preferable to be adjusted for age and sex, plus socio-economic status (low, medium, high), smoking (yes, no), physical activity and alcohol consumption (“multivariable model”). A sensitivity analysis may further be used for adjustment for the potential mediators/confounders body mass index and waist-hip ratio. The proportional hazards assumption for type-2 diabetes may be tested using Schoenfeld residuals. In addition, effect modification with formal tests may be investigated for interaction across groups defined by age (<60 years, 60-70 years, >70 years), sex, statin intake, and obesity (body mass index: <25, 25-30, >30). Principal analyses may use significance levels of two-sided P<0.05.

In context of the present invention, “identity”, “percent identity”, or “X % identical” means that nucleotide sequences have identities of at least 95%, at least 96%, at least 97%, at least 98% or at least 99% to the sequence shown herein, e.g. the sequence of SEQ ID NO: 1, wherein the higher identity values are preferred upon the lower ones. In accordance with the present invention, the term “identity/identities” or “percent identity/identities” in the context of two or more nucleic acid sequences, refers to two or more sequences that are the same, or that have a specified percentage of nucleotides that are the same (e.g., at least 95%, more preferably at least 96%, even more preferably 97%, even more preferably at least 98% or even more preferably at least 99% identity with the nucleic acid sequences of SEQ ID NO: 1, and being functional, wherein the function comprises the activity (i.e. the ability) to repress translation of the target genes of miR-122), when compared and aligned for maximum correspondence over a window of comparison, or over a designated region as measured using a sequence comparison algorithm as known in the art, or by manual alignment and visual inspection.

Preferably the described identity exists over a region that is at least about 10 nucleotides, preferably at least 15 nucleotides, more preferably at least 20 nucleotides, and most preferably all nucleotides of SEQ ID NO: 1 in length.

Those having skills in the art will know how to determine percent identity between/among sequences using, for example, algorithms such as those based on CLUSTALW computer program (Thompson, 1994, Nucl Acids Res, 2: 4673-4680) or FASTDB (Brutlag, 1990, Comp App Biosci, 6: 237-245), as known in the art. Also available to those having skills in this art are the BLAST and BLAST 2.0 algorithms (Altschul, 1997, Nucl Acids Res 25: 3389-3402; Altschul, 1993, J Mol Evol, 36: 290-300; Altschul, 1990, J Mol Biol 215: 403-410). For example, BLAST 2.0, which stands for Basic Local Alignment Search Tool BLAST (Altschul, 1997, loc. cit.; Altschul, 1993, loc. cit.; Altschul, 1990, loc. cit.), can be used to search for local sequence alignments. BLAST, as discussed above, produces alignments of both nucleotide and amino acid sequences to determine sequence similarity. Because of the local nature of the alignments, BLAST is especially useful in determining exact matches or in identifying similar sequences. Analogous computer techniques using BLAST (Altschul, 1997, loc. cit.; Altschul, 1993, loc. cit.; Altschul, 1990, loc. cit.) are used to search for identical or related molecules in nucleotide databases such as GenBank or EMBL.

The present invention is further described by reference to the following non-limiting Figures and Examples.

The Figures show:

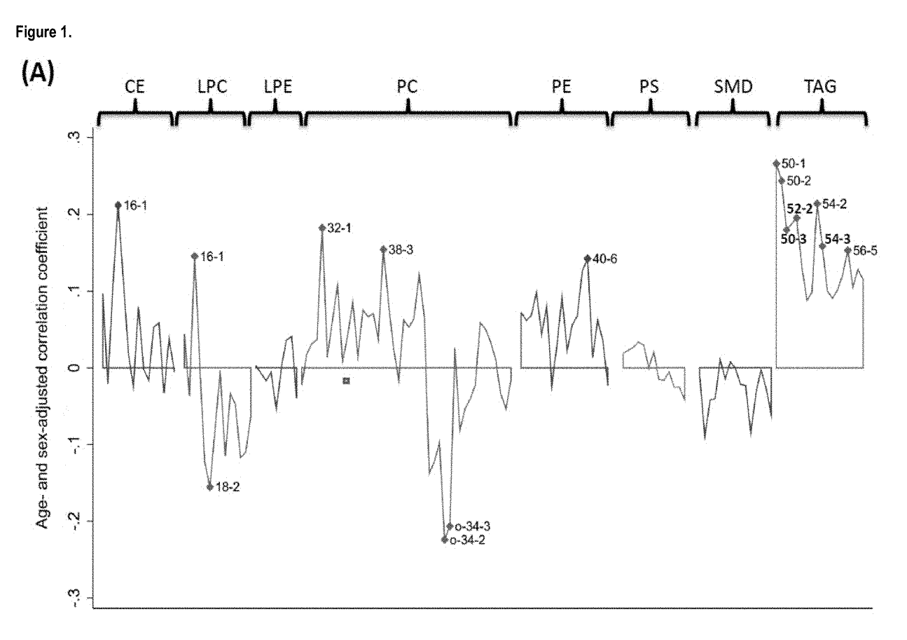

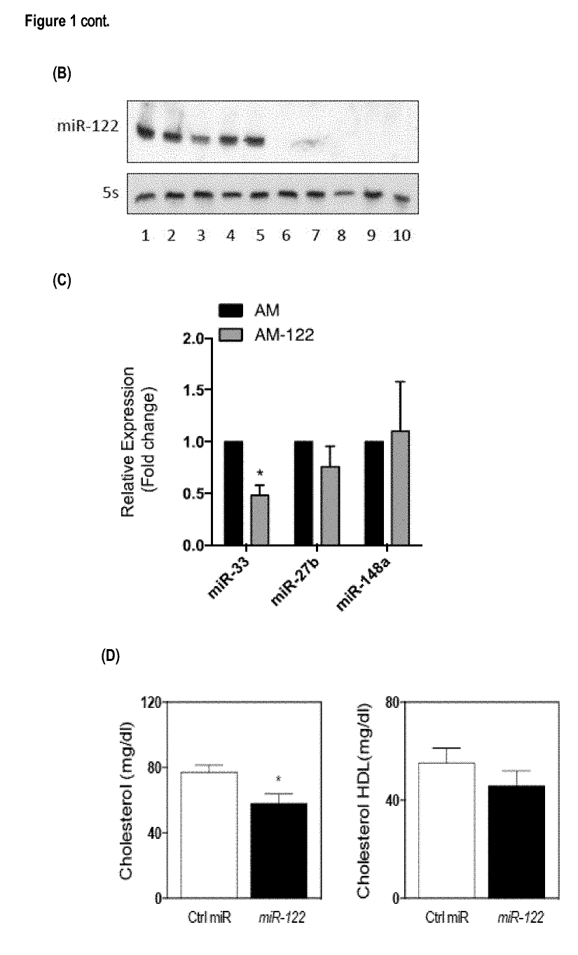

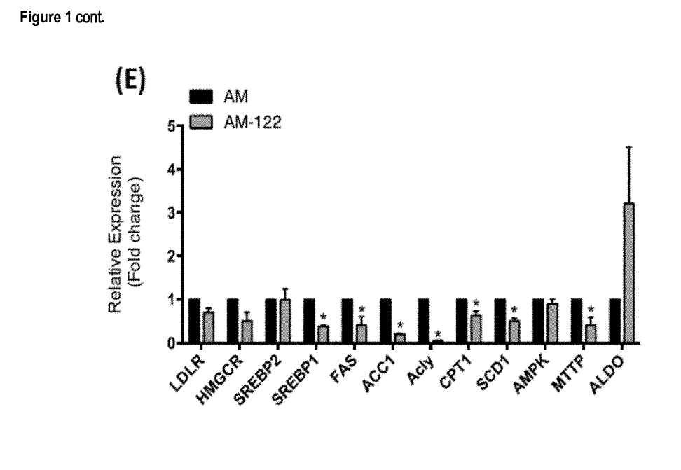

FIG. 1: Cross-sectional correlation of serum miR-122 levels with levels of lipid subspecies in the Bruneck Study (A), and consequences of antagomiR-122 injections in mice (n=5 per group), including reduction of liver miR-122 as evaluated by Northern blotting (B), effect of antagomiR-122 on the expression of other hepatic miRNAs that regulate lipoprotein metabolism, including reduction of miR-33 (C), reduction in serum cholesterol concentration upon antagomiR-122 treatment (D), and expression of genes implicated in cholesterol and lipid metabolism (E). In Panel A, the numbers indicate the fatty acid (FA) composition of the complex lipids (carbon chain length: number of double bonds) as determined by mass spectrometry; diamonds and labels indicate correlation coefficients significant after Bonferroni-correction for multiple testing. The marker label indicates the carbon chain length and the number of double bonds. Abbreviations: ACC1, acetyl-CoA carboxylase (cytosolic); Acly, ATP citrate lyase; ALDO, aldolase; AMPK, 5′ AMP-activated protein kinase; CE, cholesteryl ester; CPT1, Carnitine palmitoyltransferase 1; FAS, fatty acid synthase; HMGCR, HMG-CoA reductase; LDLR, LDL receptor; LPC, lysophosphatidylcholine; LPE, lysophosphatidylethanolamine; PC, phosphatidylcholine; PE, phosphatidylethanolamine; PS, phosphatidylserine; MTTP, microsomal triglyceride transfer protein; SCD1, Stearoyl-CoA desaturase-1; SM, sphingomyelin; SREBP, sterol regulatory element-binding protein; and TAG, triacylglycerol. *P<0.05.

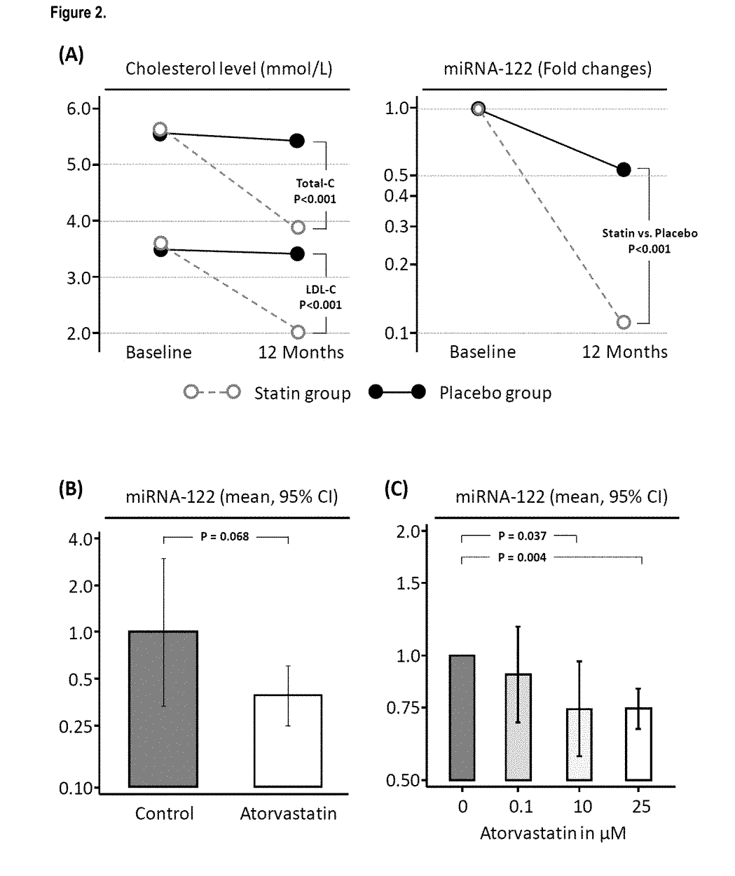

FIG. 2: Treatment with atorvastatin led to a concordant reduction in total cholesterol, LDL cholesterol, and serum miR-122 in participants of the ASCOT trial (A), reduced serum miR-122 in mice (B), and reduced miR-122 secretion from primary hepatocytes (C).

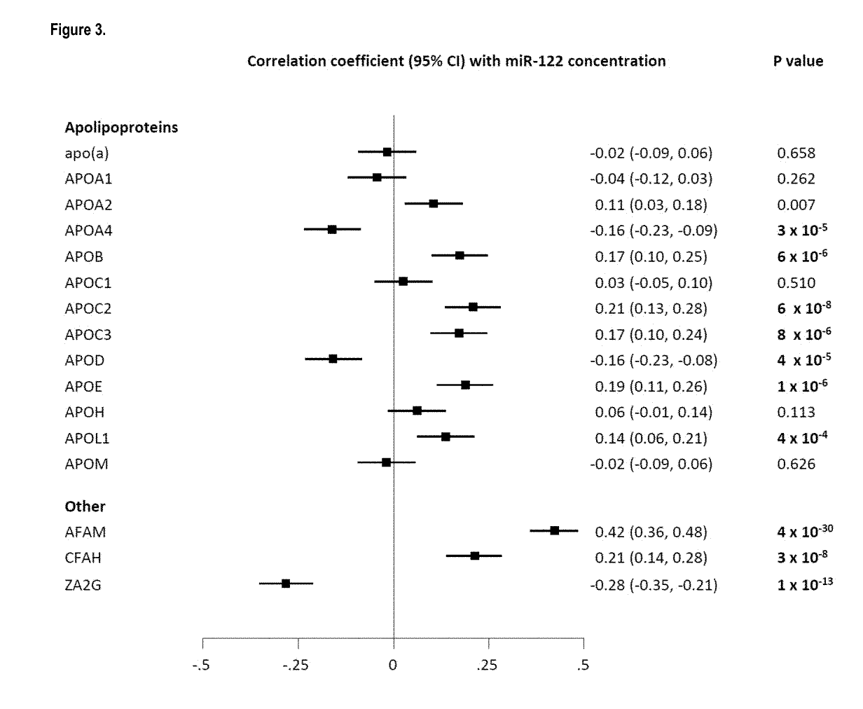

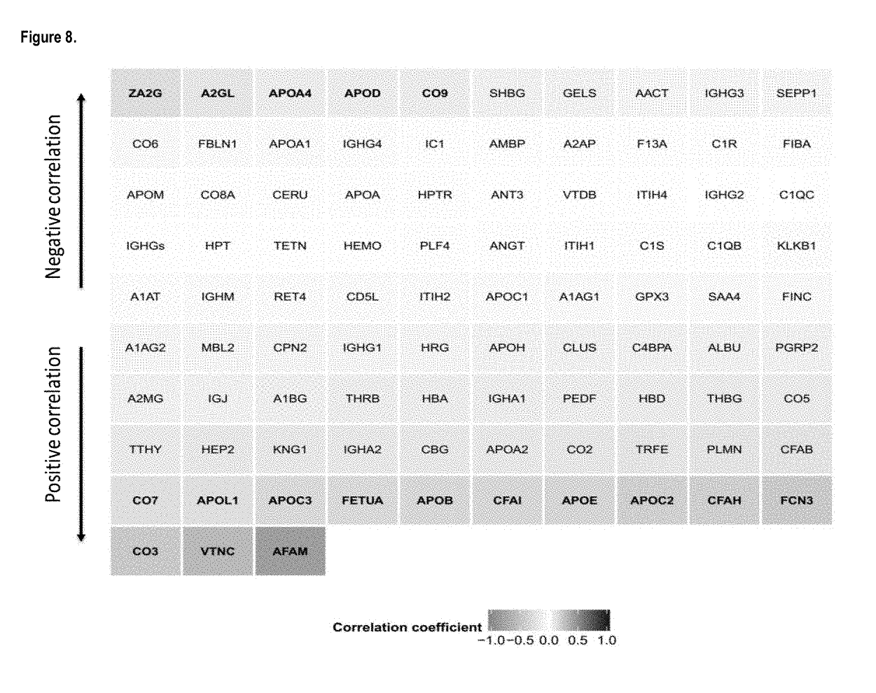

FIG. 3: Correlation of serum miR-122 levels with circulating levels of apolipoproteins and other selected proteins related to lipid metabolism in the Bruneck Study. Abbreviations: AFAM, afamin (Alpha-albumin); CFAH, complement factor H; ZA2G, zinc-alpha-2-glycoprotein. Spearman correlation coefficients were adjusted for age and sex. P values in bold were significant after Bonferroni-correction for multiple testing (the full panel of proteins are shown in FIG. 8). Other proteins were selected because of their role in lipid metabolism and their strong correlation with circulating miR-122.

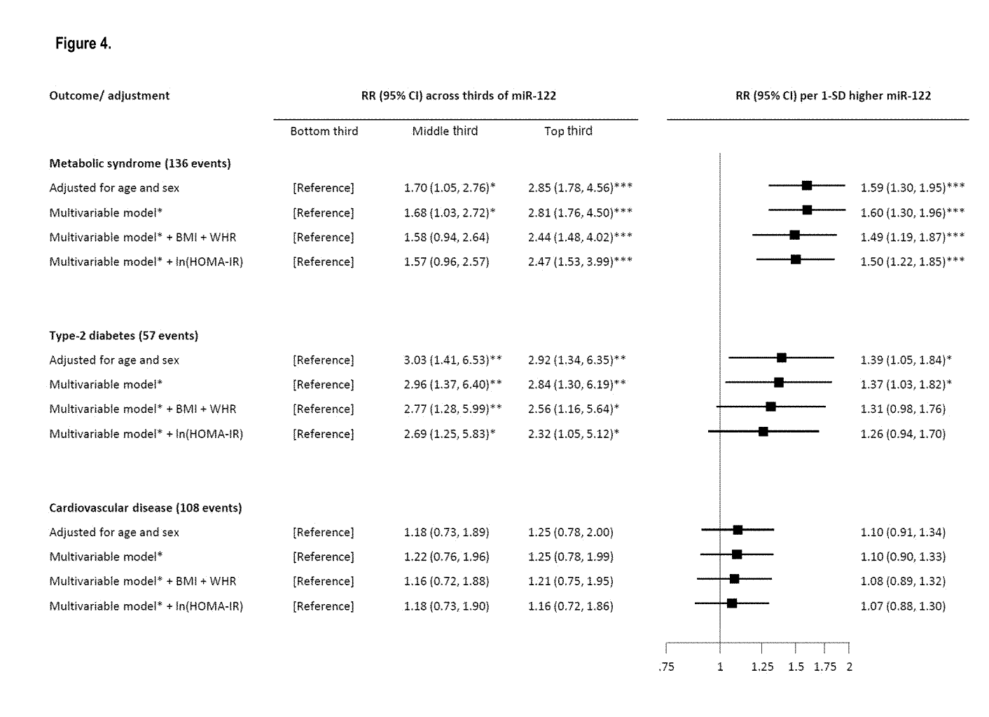

FIG. 4: Association of miR-122 with incident cardiometabolic diseases in the Bruneck Study. *The multivariable model was adjusted for age, sex, socio-economic status, smoking, physical activity, and alcohol consumption. Asterisks indicate level of significance: *P<0.05; **P<0.01; ***P<0.001.

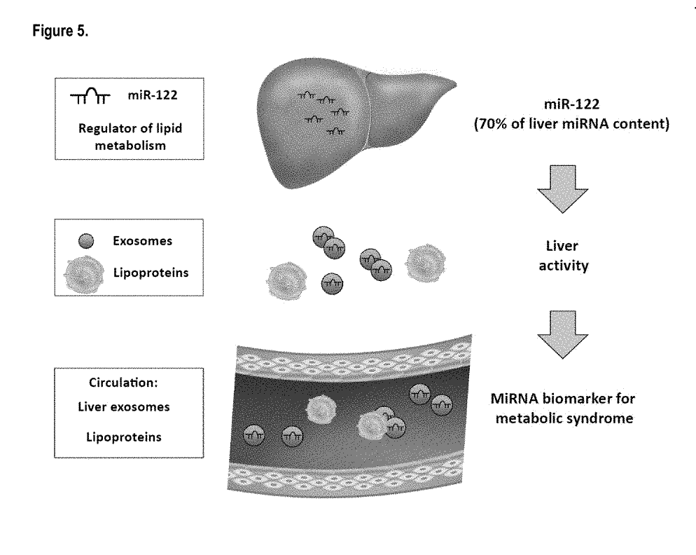

FIG. 5: Exosomes as a vehicle for liver miR-122 in circulation.

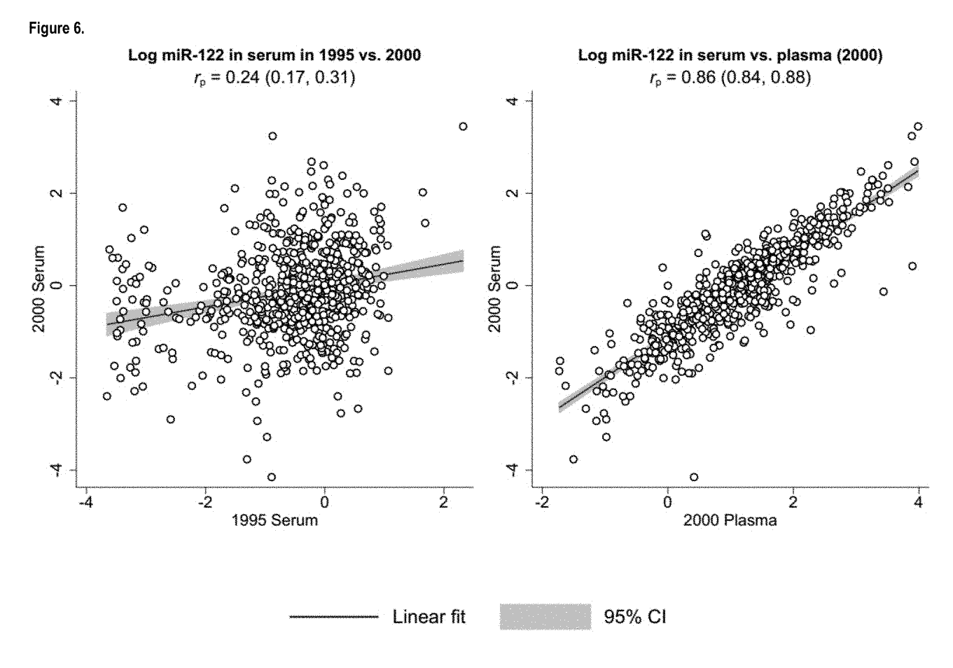

FIG. 6: Correlation of miR-122 over time and in serum vs. plasma in the Bruneck Study. MicroRNA values were log-transformed for analysis.

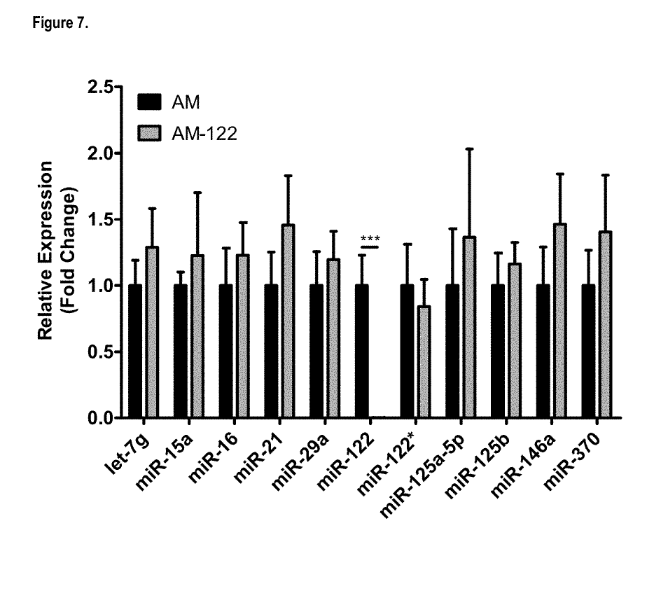

FIG. 7: Consequences of antagomiR-122 injections on miRNA expression in mouse liver as evaluated by quantitative PCR, related to FIG. 1. Data are mean (standard deviation). The relative amount of miR-122 in antagomiR-122 group was 0.13%+/−0.06% (P=6×10⁻⁴). *P<0.05; **P<0.01; ***P<0.001.

FIG. 8: Correlation of serum miR-122 with circulating levels of proteins in the Bruneck Study. Spearman correlation coefficients were adjusted for age and sex. Proteins in bold were significant after Bonferroni-correction for multiple testing. Abbreviations: A1AG1, alpha-1-acid glycoprotein 1; A1AG2, alpha-1-acid glycoprotein 2; A1AT, alpha-1-antitrypsin; A1BG, alpha-1B-glycoprotein; A2AP, alpha-2-antiplasmin; A2GL, leucine-rich alpha-2-glycoprotein; A2MG, alpha-2-macroglobulin; MCI, alpha-1-antichymotrypsin; AFAM, afamin; ALBU, albumin; AMBP, alpha-1-microglobulin; ANGT, angiotensinogen; ANT3, antithrombin-III; apo(a), apolipoprotein(a); APOA1, apolipoprotein A-I; APOA2, apolipoprotein A-II; APOA4, apolipoprotein A-IV; APOB, apolipoprotein B-100; APOC1, apolipoprotein C-I; APOC2, apolipoprotein C-II; APOC3, apolipoprotein C-Ill; APOD, apolipoprotein D; APOE, apolipoprotein E; APOH, apolipoprotein H; APOL1, apolipoprotein L1; APOM, apolipoprotein M; C1QB, complement C1q subcomponent subunit B; C1QC, complement C1q subcomponent subunit C; C1R, complement C1r subcomponent; CIS, complement C1 s subcomponent; C4BPA, C4b-binding protein alpha chain; CBG, corticosteroid-binding globulin; CD5L, CD5 antigen-like; CERU, ceruloplasmin; CFAB, complement factor B; CFAH, complement factor H; CFAI, complement factor I; CLUS, clusterin; CO2, complement C2; CO3, complement C3; CO5, complement C5; CO6, complement C6; CO7, complement C7; CO8A, complement C8 alpha chain; CO9, complement C9; CPN2, carboxypeptidase N subunit 2; F13A, coagulation factor XIII A chain; FBLN1, fibulin-1; FCN3, ficolin-3; FETUA, fetuin A; FIBA, fibrinogen alpha chain; FINC, fibronectin; GELS, gelsolin; GPX3, glutathione peroxidase 3; HBA, hemoglobin subunit alpha; HBD, hemoglobin subunit delta; HEMO, hemopexin; HEP2, heparin cofactor 2; HPT, haptoglobin; HPTR, haptoglobin-related protein; HRG, histidine-rich glycoprotein; IC1, plasma protease C1 inhibitor; IGHA1, immunoglobulin alpha 1; IGHA2, immunoglobulin alpha 2; IGHGs, immunoglobulin gamma; IGHG1, immunoglobulin gamma 1; IGHG2, immunoglobulin gamma 2; IGHG3, immunoglobulin gamma 3; IGHG4, immunoglobulin gamma 4; IGHM, immunoglobulin mu; IGJ, immunoglobulin J; ITIH1, inter-alpha-trypsin inhibitor heavy chain 1; ITIH2, inter-alpha-trypsin inhibitor heavy chain 2; ITIH4, inter-alpha-trypsin inhibitor heavy chain 4; KLKB1, kallikrein; KNG1, kininogen-1; MBL2, mannose-binding protein C; PEDF, pigment epithelium-derived factor; PGRP2, N-acetylmuramoyl-L-alanine amidase; PLF4, platelet factor 4; PLMN, plasminogen; RET4, retinol-binding protein 4; SAA4, serum amyloid A-4 protein; SEPP1, selenoprotein P; SHBG, sex hormone-binding globulin; TETN, tetranectin; THBG, thyroxine-binding globulin; THRB, prothrombin; TRFE, serotransferrin; TTHY, transthyretin; VTDB, vitamin D-binding protein; VTNC, vitronectin; ZA2G, zinc-alpha-2-glycoprotein.

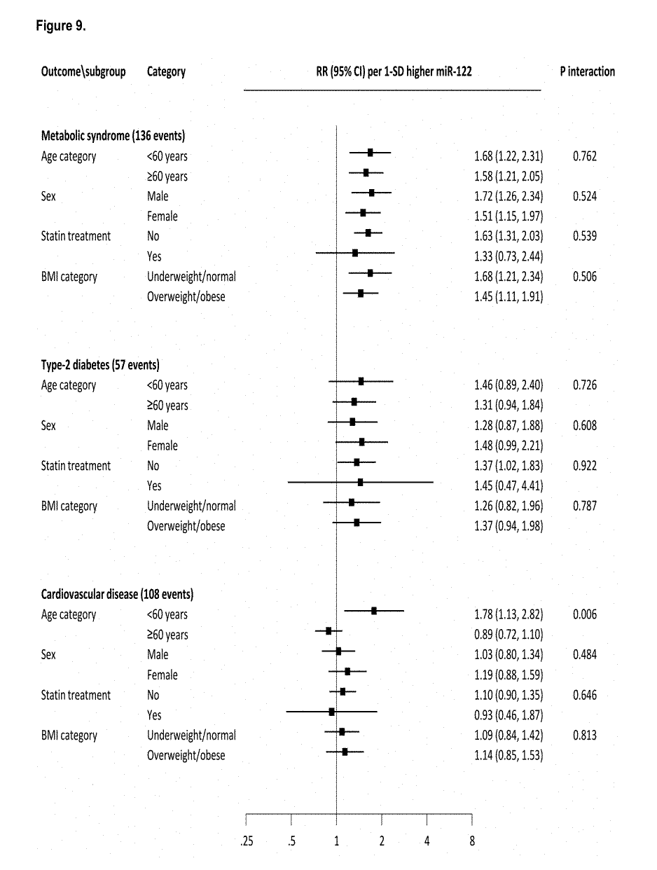

FIG. 9: Association of miR-122 with cardiometabolic diseases across clinically relevant subgroups in the Bruneck Study. Adjusted for age, sex, socio-economic status, smoking, physical activity, and alcohol consumption.

The Examples illustrate the invention.

### Example 1: Materials and Methods

The Bruneck Study

The Bruneck Study is a prospective, population-based study (Stegemann, Circulation 2014, 129: 1821-31; Kiechl, Nat Med 2013, 19: 358-63). In 1990, 1,000 individuals aged 40 to 79 years were recruited as random sample of Bruneck inhabitants and were re-examined every 5 years since, with participation rates exceeding 90% at all surveys. The present study used the 1995 survey as baseline. Full medical records are available on clinical endpoints occurring between 1995 and 2010 (1995-2005 for metabolic syndrome) for all individuals, including those who did not participate in later evaluations or died during follow up (100% follow up for clinical endpoints). Metabolic syndrome was diagnosed if three out of the five following characteristics were present (Alberti, Circulation 2009, 120: 1640-5): (i) waist circumference in men ≥102 cm and women ≥88 cm; (ii) fasting triglycerides ≥150 mg/dl or on drug treatment for elevated triglycerides (fibrates and nicotinic acids); (iii) HDL cholesterol in men <40 and women <50 mg/dl or on drug treatment for reduced HDL cholesterol (fibrates and nicotinic acids); (iv) blood pressure ≥130/≥85 mmHg or antihypertensive drug treatment in a patient with a history of hypertension; and (v) fasting glucose ≥100 mg/dl or on drug treatment for elevated glucose. T2DM was diagnosed according to 1997 American Diabetes Association criteria or if the participant had a clinical diagnosis of T2DM and received anti-diabetic treatment. CVD was defined as myocardial infarction, stroke, or vascular death (Willeit, Arterioscler Thromb Vasc Biol 2010, 30: 1649-56). Fatal and nonfatal myocardial infarction were deemed confirmed when World Health Organization criteria for definite disease status were met. Ischemic stroke and transient ischemic attacks were classified according to the criteria of the National Survey of Stroke. Self-report of disease was always confirmed by reviewing the medical records of the participant's general practitioners and Bruneck Hospital. The Bruneck Study protocol was approved by the ethics committees of Bolzano and Verona and all study participants gave their written informed consent before taking part. Risk factors were ascertained by validated standard procedures as previously described (Willeit, Arterioscler Thromb Vasc Biol 2010, 30: 1649-56; Kiechl, N Engl J Med 2002, 347: 185-92). Socioeconomic status was defined on a three-category scale (low, medium or high) on the basis of information on occupational status and educational level of the person with the highest income in the household. High socioeconomic status was assumed if the participant had ≥12 years of education or an occupation with an average monthly income ≥$2,000 (baseline salary before tax). Low socioeconomic status was defined by ≤8 years of education or an average monthly income ≤$1,000. Physical activity was assessed using the validated Baecke Score (Baecke, Am J Clin Nutr 1982, 36: 936-42). Waist and hip circumferences were assessed with a plastic tape measure at the levels of the umbilicus and the greater trochanters respectively. Blood samples were taken after an overnight fast. Lipidomics profiling was performed with mass spectrometry, which allowed quantification of 135 distinct lipid species (Stegemann, Circulation 2014, 129: 1821-31; Shah, Circulation 2012, 126: 1110-20). HbA1c was quantified using high performance liquid chromatography (DCCT-aligned assay). The degree of insulin resistance by homeostasis model assessment (HOMA-IR) was estimated using the formula fasting plasma glucose in mmol/l×fasting serum insulin in mU/I divided by 22.5 (Bonora, Int J Obes Relat Metab Disord 2003, 27: 1283-9), with higher HOMA-IR values indicating higher insulin resistance. MiR-122 was measured in serum taken at the 1995 examination (n=810) as well as in serum and plasma taken at the 2000 examination (n=695).

Multiple Reaction Monitoring (MRM) for Plasma Proteins

PlasmaDive kits (Biognosys AG) were used to profile plasma proteins in the Bruneck Study. Plasma samples were processed according to the manufacturer's instructions with one exception: peptide standards were spiked in before and not after tryptic digestion and C18 clean-up. Briefly, 10 μl of plasma samples were denatured, reduced and alkylated. 20 μg of proteins were spiked with 100 authentic heavy peptide standards. Seven proteins were below the limit of detection. An in-solution digestion was performed overnight. After solid phase extraction with C18 spin columns (96-well format, Harvard apparatus), the eluted peptides were dried using a SpeedVac (Thermo) and resuspended in 40 μl of liquid chromatography (LC) solution. The samples were analysed on an Agilent 1290 LC system interfaced to an Agilent 6495 Triple Quadrupole MS. 10 μl samples were directly injected onto a 25 cm column (AdvanceBio Peptide Map 2.1×250 mm) and separated over a 23 min gradient at 300 μl/min. The data were analysed using Skyline software version 3.1 (MacCross Lab) and protein concentrations were calculated using the heavy/light (H/L) ratio. During continuous operation over 2 weeks, the inter-day relative standard deviation (RSD) was <20% and <5% without and with adjustment for the peak area of the authentic standard peptides, respectively.

The ASCOT Trial

The Anglo-Scandinavian Cardiac Outcomes Trial (ASCOT) trial is a double-blind randomized 2×2 factorial study of blood-pressure lowering and lipid-lowering treatment (Sever, Lancet 2003, 361: 1149-58; Poulter, Lancet 2005, 366: 907-13). A total of 14,412 patients (aged 40-79 years) were randomised into ASCOT between 1998 and 2000 using a computer-generated optimum allocation mechanism blinded for any person involved in the undertaking of the study. Patients randomised into the lipid-lowering arm had low to moderate cholesterol levels (serum total cholesterol≤6.5 mmol/1) and were assigned atorvastatin (10 mg/day) or placebo. Serum miR-122 levels were measured at baseline and 1 year (median 13 [range 12 to 16] months) post initiation of statin treatment in randomized individuals of European ethnicity without T2DM who participated in the hypertension-associated cardiovascular disease (HACVD) sub-study of ASCOT (n=155: 73 in intervention and 82 in placebo group) (Stanton, J Hum Hypertens 2001, 15 Suppl 1: S13-8).

AntagomiR Treatment of Mice

Mice were injected intraperitoneally with antagomiR-122 and control antagomiRs (65 mg/kg) on three consecutive days as previously described (Zampetaki, Circ Res 2014, 115: 857-66). AntagomiRs were purchased from Fidelity Systems with the following sequences: antagomiR-122-C*A*AACACCAUUGUCACACU*C*C*A*Chol*-T; controls-A*A*GGCAAGCUGACCUGAA*G*U*U*Chol-T. Mice were sacrificed at day 7. Liver and serum samples were harvested for analysis.

Northern Blot Analysis

MiRNA expression was assessed by Northern blot analysis as previously described (Suárez, Circ Res 2007, 100: 1164-73). Briefly, total RNA (5 μg) was separated on a 15% acrylamide TBE 8M urea gel and blotted onto a Hybond N+ nylon filter (Amersham Biosciences). DNA oligonucleotides complementary to mature miR-122 (5′-AAACACCATTGTCACACTCCA-3′) were end-labeled with [α-³²P] ATP and 14 polynucleotide kinase (New England Biolabs) to generate high-specific activity probes. Hybridization was carried out according to the ExpressHyb (Clontech) protocol. Following overnight membrane hybridization with specific radiolabeled probes, membranes were washed once for 30 min at 42° C. in 4×SSC/0:5% SDS and subjected to autoradiography. Blots were re-probed for 5S rRNA to control for equal loading: 5′-CAGGCCCGACCCTGCTTAGCTTCCGAGAGATCAGACGAGAT-3′

RNA Isolation and Quantitative Real-Time PCR (qRT-PCR)

Total RNA was isolated from liver and hepatocyte samples using TRIzol reagent (Invitrogen) according to the manufacturer's protocol. For mRNA quantification, cDNA was synthesized using iScript RT Supermix (Bio-Rad), following the manufacturer's protocol. qRT-PCR analysis was performed in triplicate using iQ SYBR green Supermix (BioRad) on an iCycler Real-Time Detection System (Eppendorf). The mRNA level was normalized to GAPDH or 18S as a house keeping gene (see primer sequence in Table 2). For miRNA quantification, total RNA was reverse transcribed using the miScript II RT Kit (Qiagen). Primers specific for mouse miR-122, miR-27b, miR148a and miR-33a (Qiagen) were used and values normalized to SNORD68 (Qiagen). For mouse tissues, total liver RNA from WT mice fed a HFD were isolated using the Bullet Blender Homogenizer (Next Advance) in TRIzol. 1 μg of total RNA was reverse transcribed and gene/miRNA expression assessed as above.

Measurements of Total and HDL Cholesterol in Mice

Blood samples were collected by retro-orbital venous plexus puncture after a 12 h overnight fast for lipid analysis. Plasma was separated by centrifugation and stored at 4° C. Total and HDL cholesterol in each sample was enzymatically measured using the T-Cholesterol and HDL-C Assay Kits (Wako Diagnostics).

Statin Treatment of Mice

Six week old, female C57BI/6 mice were purchased from Harlan Laboratories (San Pietro al Natisone, Italy) and housed at 22° C. under a 12 h light/dark cycle under specific pathogen-free conditions with ad libitum access to chow and water. Mice were injected once a day with 20 mg/kg atorvastatin intraperitoneally (Sigma Aldrich, Taufkirchen, Germany) for five days. Mice were sacrificed on day 5. Serum was collected by cardiac puncture. The liver was perfused with ice-cold phosphate-buffered saline and tissue specimens from the left lower lobe were either snap frozen or placed in RNAlater (Qiagen, Hilden, Germany) until further processing.

Statin Treatment of Primary Hepatocytes

For in vitro experiments, primary mouse hepatocytes were isolated as previously described (Moschen, Hepatology 2011, 54: 675-86). Briefly, mice were anesthetized, the abdomen was opened and liver, vena cava, and portal vein were prepared. The liver was perfused regressively from the abdominal vena cava, via the liver veins (the vena cava proximal to the liver veins was occluded with a microclamp) to the portal vein using a peristaltic pump (Bio-Rad, Hercules, Calif.). The liver was first perfused using liver perfusion medium 1 supplemented with 0.1 mM EGTA at a flow rate of 7 ml/min for 10 min. Thereafter, liver perfusion medium 2 containing 30 μg/ml Liberase™ (Roche, Mannheim, Germany) was used at a flow rate of 3.5 ml/min for another 10 min. The liver was removed carefully and transferred into a petri-dish containing L-15 medium (Gibco, Carlsbad, Calif.). The capsule was incised and the resulting cell suspension was passed through a 100 μm cell strainer. Hepatocytes were sedimented by low-speed centrifugation at 30×g for 3 min. Purity and viability were >90% after an isodensity Percoll centrifugation. 1.3×10⁵ hepatocytes per cm² were seeded in William's E medium supplemented with 10% FCS and penicillin/streptomycin on collagen-coated 6-well plates. After resting cells overnight, hepatocytes were exposed to 0.1, 10, and 25 μM atorvastatin for 24 h. Supernatants were harvested and cells were lysed in Buffer RLT. Both were stored at −80° C. until further workup.

MiRNA-122 Measurements with qRT-PCR

MiR-122 was measured as previously described (Willeit, Circ Res 2013, 112: 595-600; Zampetaki, Circ Res 2010, 107: 810-7). Briefly, miRNAs were extracted using the miRNeasy kit (Qiagen, Hilden, Germany). For plasma, serum or cell culture supernatants, a fixed volume of 3 μl of the 25 μl RNA eluate was used as input for reverse transcription (RT) reactions. For RNA from cells or tissue, 100 ng input material was used for RT. MiRNAs were reversely transcribed using Megaplex Primer Pools (Human Pools A version 2.1 or Rodent Pool A, Life Technologies, Darmstadt, Germany) and products were further amplified using Megaplex PreAmp Primers (Primers A v2.1). Both RT and PreAmp products were stored at −20° C. Taqman miRNA assays were used to assess the expression of individual miRNAs. Diluted pre-amplification product (0.5 μl) or RT product (corresponding to 0.45 ng input) were combined with 0.25 μl Taqman microRNA assay (20×) (Life Technologies) and 2.5 μl Taqman Universal PCR Master Mix No AmpErase UNG (2×) to a final volume of 5 μl. A qRT-PCR was performed on an Applied Biosystems 7900HT thermocycler at 95° C. for 10 min, followed by 40 cycles of 95° C. for 15 s and 60° C. for 1 min. All samples were run in duplicates. Laboratory technicians were blinded to the participants' disease status. Relative quantification was performed using the software SDS2.2 (Life Technologies). U6 and exogenous C. elegans spike-in control (Cel-miR-39) were used for normalisation purposes.

Gene Expression Analysis with qRT-PCR

Total RNA was reversely transcribed using the high-capacity cDNA RT kit (Life Technologies). RT product (corresponding to 6.75 ng input RNA), 0.25 μl Taqman gene expression assay and 2.5 μl Taqman Universal PCR Master Mix No AmpErase UNG (2×) were combined in a total volume of 5 μl. Cycling conditions were identical to miRNA analysis. GAPDH was used as a normalisation control.

Statistics

The statistical analysis was conducted according to a pre-specified analysis plan. MiR-122 values were log-transformed for analysis. Cross-sectional associations of miR-122 levels with other participant characteristics were quantified using spearman correlation coefficients and linear regression models adjusted for age and sex. The prospective analysis used Cox proportional hazard regression with updated covariates for CVD and T2DM, and pooled logistic regression (D'Agostino, Stat Med 1990, 9: 1501-15) for metabolic syndrome. Both techniques make full use of the repeat measurements of miR-122 available at the 1995 and 2000 examination. Hazard ratios and odds ratios were assumed to represent the same measure of relative risk and are collectively described as risk ratios (RR). Participants with prevalent disease were excluded from the respective analyses. Models were adjusted for age and sex, plus socio-economic status (low, medium, high), smoking (yes, no), physical activity and alcohol consumption (“multivariable model”). A sensitivity analysis further adjusted for the potential mediators/confounders body mass index and waist-hip ratio. The proportional hazards assumption for CVD and T2DM was tested using Schoenfeld residuals and was met. We investigated effect modification with formal tests for interaction across groups defined by age (<60 years, 60-70 years, >70 years), sex, statin intake, and obesity (body mass index: <25, 25-30, >30). Principal analyses used significance levels of two-sided P<0.05. Exploratory analyses used Bonferroni-corrected P values to limit the risk of false-positive results (i.e. 0.00037 for analyses of lipid subspecies; 0.00054 for proteins; 0.0042 for interaction tests). Analyses were performed using Stata software, version 12.1. Study methods and findings are reported according to the STROBE guidelines.

Study Approval

The Bruneck Study protocol was approved by the local ethic committee of Bolzano (‘Comitato etico del comprensorio sanitario di Bolzano’; approval number 28-2010). The ASCOT trial protocol was approved by central and regional ethics review boards in the UK, and by national ethics and statutory bodies in Ireland and the Nordic countries. Animal experiments were approved by the Austrian authorities (licensed to A. R. Moschen No BMWF-66.011/0040-II/10b/2009) and UK authorities (licensed to Q. Xu No. PPL70/7266). Written informed consent was received from Bruneck and ASCOT participants prior to inclusion in the studies.

### Example 2: Circulating MicroRNA-122 is Associated with Incident Metabolic Syndrome and Type-2 Diabetes

Summary of the Example

In the prospective population-based Bruneck Study, circulating miR-122 was associated with insulin resistance, obesity, metabolic syndrome, diabetes, and an adverse lipid profile. Using mass spectrometry, 135 lipid subspecies and 93 plasma proteins were quantified. MiR-122 was closely related to levels of afamin, zinc-alpha-2-glycoprotein, complement-factor H, and several apolipoproteins, in particular apoB, apoC2, apoC3, and apoE. In the Anglo-Scandinavian Cardiac Outcomes Trial (ASCOT, n=155), atorvastatin treatment reduced circulating miR-122 levels. A similar statin response was observed in mice and cultured hepatocytes. Over up to 15-years of follow-up (1995-2010), incidence of cardiometabolic diseases was recorded in the Bruneck Study (follow-up for clinical events: 100%). Risk ratios adjusted for age, sex, socio-economic status, smoking, physical activity, and alcohol consumption per 1-SD higher log miR-122 were: 1.60 (1.30-1.96; P<0.001) for metabolic syndrome, 1.37 (1.03-1.82; P=0.021) for type-2 diabetes, and 1.10 (0.90-1.33; P=0.330) for cardiovascular disease.

Conclusions:

Circulating miR-122 levels are strongly associated with hepatic metabolism and with the risk of developing metabolic syndrome and diabetes in the general population.

Detailed Results

Correlates of miR-122 in the Bruneck Study

MiR-122 was successfully quantified in 810 out of 826 participants of the Bruneck Study; their baseline characteristics are summarised in Table 1. The mean age of study participants was 63 years (SD, 11) and half were female. In an analysis of repeat measurements of miR-122 taken 5 years apart, the within-person correlation was 0.24 (95% confidence interval: 0.17-0.31; FIG. 6). The correlation coefficient between miR-122 levels in plasma and in serum was 0.86 (0.84-0.88; FIG. 6). In an age- and sex-adjusted analysis, miR-122 levels were associated with levels of liver enzymes, insulin sensitivity, adiposity, major lipids (triglycerides, LDL- and HDL-C), and prevalence of T2DM and metabolic syndrome (Table 1). Compared to their healthy counterparts, circulating miR-122 was 214% (70-481%) higher in participants with T2DM and 160% (76-285%) higher in participants with metabolic syndrome.

In particular, the amount of miR-122 has been determined as means and interquartile ranges. Persons with a risk for developing metabolic syndrome have been shown to have a median miR-122 value of 1.05 (interquartile range: 0.65-2.93). Similarly, persons with a risk for developing type-2 diabetes have a median miR-122 value of 0.94 (interquartile range: 0.55-1.66). In contrast, persons with no risk for either of the two diseases have a median miR-122 value of 0.65 (interquartile range: 0.34-1.12). In addition, it has been found that in persons that have a manifested metabolic syndrome the amount of miR-122 is increased by 160% (i.e. to a value of 260%) as compared to the healthy reference population. Similarly, in persons that have manifested type-2 diabetes the amount of miR-122 is increased by 214% (i.e. to a value of 314%) as compared to the healthy reference population. Surprisingly, already 5 years prior to manifestation of these diseases, the affected persons showed an increased level of miR-122. In particular, 5 years before manifestation of the metabolic syndrome the amount of miR-122 was already increased by 104% (i.e. to a value of 204%) as compare to the healthy reference population. Similarly, 5 years before manifestation of type-2 diabetes, the miR-122 level was already increased by 49% (i.e. to a value of 149%) as compared to the healthy reference population.

MiR-122 levels were positively associated with triglycerides, more weakly with body mass index and waist-hip ratio, and inversely with HDL-cholesterol.

TABLE 1
Baseline characteristics and cross-sectional correlates of miR-122 in the Bruneck Study.

| | | | | |
|---|---|---|---|---|
| | | | Difference in miR-122 per SD or | |
| | | Correlation coefficient | compared to reference | |
| Variable | Mean (SD) or % | (95% CI)* | (95% CI)* | P value |

Questionnaire-based

| | | | | | | | |
|---|---|---|---|---|---|---|---|
| Age, years | 63 | (11) | −0.01 | (−0.08, 0.06) | −3% | (−19, 17%) | 0.753 |
| Physical activity, Baeke score | 2.3 | (.9) | 0.00 | (−0.07, 0.07) | 1% | (−18, 24%) | 0.932 |
| Alcohol consumption, g/d | 24 | (31) | −0.00 | (−0.07, 0.07) | −1% | (−21, 23%) | 0.919 |

| Sex | | | | | |
|---|---|---|---|---|---|
| Male | 50% | NA | [Reference] | | |
| Female | 50% | NA | 31% | (−10, 89%) | 0.153 |
| Current smoker | | | | | |
| No | 80% | NA | [Reference] | | |
| Yes | 20% | NA | −22% | (−51, 26%) | 0.314 |
| Socioeconomic status | | | | | 0.022 |
| Low | 61% | NA | [Reference] | | |
| Middle | 22% | NA | −30% | (−56, 14%) | 0.150 |
| High | 17% | NA | 65% | (−2, 177%) | 0.058 |
| Prevalent CVD | | | | | |
| No | 89% | NA | [Reference] | | |
| Yes | 11% | NA | 1% | (−46, 88%) | 0.969 |
| Statin treatment | | | | | |
| No | 97% | NA | [Reference] | | |
| Yes | 3% | NA | −12% | (−69, 151%) | 0.808 |

Liver function enzymes

| | | | | | | | |
|---|---|---|---|---|---|---|---|
| Alanine transaminase, U/I | 23 | (13) | 0.27 | (0.20, 0.33) | 112% | (76, 155%) | <0.001 |
| Aspartate aminotransferase, U/I | 24 | (9.3) | 0.22 | (0.15, 0.28) | 81% | (51, 118%) | <0.001 |
| Log GGT, U/I | 3.3 | (.68) | 0.41 | (0.35, 0.46) | 128% | (90, 173%) | <0.001 |

Major lipid species

| | | | | | | | |
|---|---|---|---|---|---|---|---|
| Total cholesterol, mg/dl | 230 | (43) | 0.06 | (−0.01, 0.13) | 19% | (−1, 43%) | 0.070 |
| LDL cholesterol, mg/dl | 145 | (38) | 0.07 | (0.00, 0.14) | 21% | (1, 46%) | 0.043 |
| HDL cholesterol, mg/dl | 59 | (16) | −0.15 | (−0.22, −0.09) | −34% | (−45, −21%) | <0.001 |
| Log triglycerides, mg/dl | 4.8 | (.5) | 0.18 | (0.11, 0.24) | 62% | (35, 94%) | <0.001 |
| Lipoprotein(a), mg/dl | 26 | (33) | 0.01 | (−0.06, 0.08) | 3% | (−15, 23%) | 0.791 |

Markers of inflammation

| | | | | | | | |
|---|---|---|---|---|---|---|---|
| Log hsCRP, mg/l | −1.7 | (1) | 0.13 | (0.06, 0.19) | 42% | (17, 71%) | <0.001 |
| Fibrinogen, mg/dl | 289 | (76) | 0.07 | (0.00, 0.14) | 22% | (1, 48%) | 0.044 |
| Log interleukin-6, pg/ml | 1.4 | (.99) | 0.03 | (−0.04, 0.10) | 8% | (−10, 31%) | 0.415 |

Adiposity measures

| | | | | | | | |
|---|---|---|---|---|---|---|---|
| Body mass index, kg/m² | 26 | (3.9) | 0.13 | (0.06, 0.20) | 41% | (17, 69%) | <0.001 |
| Waist-hip ratio | .93 | (.072) | 0.12 | (0.05, 0.19) | 44% | (17, 76%) | <0.001 |

Dysglycaemia

| | | | | | | | |
|---|---|---|---|---|---|---|---|
| Fasting plasma glucose, mg/dl | 102 | (25) | 0.08 | (0.01, 0.14) | 23% | (2, 48%) | 0.030 |
| HbA1c, % | 5.6 | (1.8) | 0.01 | (−0.06, 0.08) | 4% | (−14, 25%) | 0.704 |
| Log HOMA-IR | 1.1 | (.6) | 0.19 | (0.12, 0.26) | 67% | (39, 101%) | <0.001 |

| Prevalent diabetes | | | | | |
|---|---|---|---|---|---|
| No | 90% | NA | [Reference] | | |
| Yes | 10% | NA | 214% | (70, 481%) | <0.001 |

Prevalent metabolic syndrome

| No. of components | | | | | |
|---|---|---|---|---|---|
| Zero | 9% | NA | [Reference] | | |
| One | 28% | NA | 49% | (−28, 207%) | 0.278 |
| Two | 30% | NA | 35% | (−34, 177%) | 0.409 |
| Three | 21% | NA | 232% | (56, 607%) | 0.002 |
| Four | 9% | NA | 324% | (74, 938%) | 0.002 |
| Five | 4% | NA | 234% | (4, 975%) | 0.043 |
| More than three components | | | | | |
| No | 67% | NA | [Reference] | | |
| Yes | 33% | NA | 160% | (76, 285%) | <0.001 |

*Adjusted for age and sex.
NA = not applicable.

MiR-122 and Lipidomic Profiles

Of the 135 lipid subspecies measured in the Bruneck Study, miR-122 showed a specific association with lipid subspecies containing saturated and monounsaturated fatty acids that can be derived from hepatic de novo lipogenesis (FIG. 1A). This finding in the general population is supported by the role of miR-122 in the regulation of liver metabolism as demonstrated by experiments using injections of antagomiR-122 in mice. We achieved an almost complete inhibition of miR-122 expression (FIG. 1B and FIG. 8). Interestingly, the hepatic expression of miR-33, a miRNA that controls the expression of numerous genes involved in lipid metabolism, was attenuated in mice treated with antagomiR-122. The expression of other miRNAs associated with lipid metabolism including miR-27b and miR-148a was similar in both groups of mice (FIG. 1C). Consistent with previous reports (Esau, Cell Metab 2006, 3: 87-98; Krützfeldt, Nature 2005, 438: 685-9; Elmén, Nature 2008, 452: 896-9; Lanford, Science 2010, 327: 198-201), treatment with antagomiR-122 resulted in lower cholesterol levels (FIG. 1D). Further, we observed down-regulation of a range of genes implicated in lipid metabolism, specifically in de novo lipogenesis and lipoprotein assembly (ACC1, CTP1, FAS, SCD1, MTTP, SREBP1; FIG. 1E).

MiR-122 and Proteomic Profiles

The lipidomics measurements in the Bruneck study were complemented by an assessment of plasma proteins, over 4 orders of magnitude in abundance by MS. The protein panel covers a wide range of apolipoproteins, complement and coagulation factors to provide insights into how miR-122 effects on lipid metabolism may mediate cardiometabolic risk. MiR-122 was most strongly associated with afamin (age- and sex-adjusted correlation coefficient: +0.42; P=4×10⁻³⁰) and zinc-alpha-2-glycoprotein (−0.28; P=10⁻¹³) (FIG. 3). Focused analyses of apolipoproteins revealed significant positive correlations with APOB, APOC2, APOC3, and APOE, and significant inverse correlations with APOA4 and APOD (FIG. 3). Correlations for all proteins are provided in FIG. 8.

Effect of Statins on miR-122 Levels

To further assess the interplay of hepatic lipid metabolism and circulating miR-122 levels, we used samples from the Hypertension Associated Cardiovascular Disease (HACVD) sub-study of the ASCOT trial. Treatment with atorvastatin led to a reduction in concentrations of total cholesterol, low-density lipoprotein cholesterol (LDL-C), and serum miR-122 levels (FIG. 2A) (all P<0.001), but not of other miRNAs quantified in the same samples (data not shown). Similar effects were observed in serum of mice treated with atorvastatin, and in culture media of primary hepatocytes incubated with 10 and 25 μM atorvastatin (FIGS. 2B and 2C).

MiR-122 and Incident Cardiometabolic Diseases in the Bruneck Study

In the Bruneck Study, we recorded 136 incident events of metabolic syndrome, 57 events of T2DM, and 108 events of CVD. Age and sex-adjusted risk ratios comparing top vs. bottom third of miR-122 levels were: 2.85 (1.78-4.56; P<0.001) for metabolic syndrome, 2.92 (1.34-6.35; P=0.007) for T2DM, and 1.25 (0.78-2.00; P=0.347) for CVD (FIG. 4). Risk ratios per 1-SD higher log miR-122 were: 1.60 (1.30-1.96; P<0.001) for metabolic syndrome, 1.37 (1.03-1.82; P=0.021) for T2DM, and 1.10 (0.90-1.33; P=0.330) for CVD (FIG. 4). Risk ratios remained virtually identical when further adjusting for socio-economic status, smoking, physical activity, and alcohol consumption, but somewhat attenuated upon further adjustment for body mass index and waist-hip ratio and for In HOMA-IR (FIG. 4). We observed broadly similar results for women and men, statin intake, and clinical categories of adiposity, but a possibly stronger association with incident CVD in participants aged <60 years compared to their older counterparts (P=0.006) (FIG. 9).

Discussion

In the present study, we use a multi-dimensional 'omics approach (lipidomics and proteomics) in a population-based study to identify metabolic signatures associated with miR-122. We report a number of important and entirely novel results based on miRNA measurements in >3000 human blood samples combined with experimental follow-up to provide a mechanistic context. First, circulating miR-122 levels are elevated in people with prevalent cardiometabolic disease and correlate strongly with lipid species that can be produced by hepatic de novo lipogenesis. In a prospective setting, elevated serum levels of miR-122 antedate the manifestation of metabolic syndrome and T2DM, but not CVD. Second, serum levels of miR-122 positively correlated with major lipids (triglycerides, LDL- and HDL-C) in the general community and substantially decline with cholesterol lowering through statin therapy (atorvastatin 10 mg). We further corroborate this observation by in vitro and in vivo experiments demonstrating a reduction of miR-122 in the supernatant of atorvastatin-challenged human hepatocytes and in serum of atorvastatin-treated wild-type mice and confirmed miR-122 effects on enzymes involved in lipid metabolism in the liver. Overall, we provide strong evidence for circulating miR-122 being a marker of hepatic lipid and glucose metabolism.

System-Wide Relations of Circulating miR-122

MiR-122, which is primarily expressed in the liver (Lagos-Quintana, Curr Biol 2002, 12: 735-9), has been suggested to regulate the expression of various genes associated with cholesterol and fatty acid metabolism (Rottiers, Nat Rev Mol Cell Biol 2012, 13: 239-50). In mice, inhibition of miR-122 using antisense oligonucleotides and antagomiRs led to markedly lower plasma cholesterol levels, halted hepatic lipid synthesis, and enhanced hepatic fatty acid oxidation (Esau, Cell Metab 2006, 3: 87-98; Krützfeldt, Nature 2005, 438: 685-9). Two studies in non-human primates reported similar reductions in cholesterol (Elmén, Nature 2008, 452: 896-9; Lanford, Science 2010, 327: 198-201). In line with these reports, our study showed that a selective inhibition of miR-122 using antagomiR-122 (with some cross-talk with miR-33) led to a down-regulation of enzymes implicated in lipid metabolism. Furthermore, in the Bruneck Study, high circulating miR-122 levels in the circulation were generally associated with an adverse lipid profile, which we and others have previously shown to be related to CVD (Stegemann, Circulation 2014, 129: 1821-31) and incident T2DM (Rhee, J Clin Invest 2011, 121: 1402-11) (strong positive correlations with cholesteryl esters and triacylglycerols, strong inverse correlations with lysophosphatidylcholines). The positive correlation of miR-122 with lipids containing saturated and monounsaturated fatty acids with reduced carbon chain length together with the positive correlation with apolipoproteins found on very-low density lipoprotein (VLDL) (apoB100, apoC1, apoC2, apoE) support a link between miR-122 and hepatic de novo lipogenesis or VLDL secretion. This is further corroborated by the notion that miR-122 knockout mice express less microsomal triglyceride transfer protein (MTTP), an essential enzyme that regulates the biogenesis of lipoproteins (Wen, J Clin Invest 2012, 122: 2773-6). In contrast, circulating miR-122 showed inverse associations with plasma ApoD (present mainly in HDL) and apoA4 (a major component of HDL and chylomicrons). Moreover, our comprehensive assessment of plasma proteins returned a positive correlation with afamin, which was previously linked to prevalent and incident metabolic syndrome and all its components (Kronenberg, Circ Cardiovasc Genet 2014, 7: 822-9), a positive correlation with complement factor H, a protein that binds malondialdehyde epitopes and protects from oxidative stress (Weismann, Nature 2011, 478: 76-81), and an inverse correlation with zinc-alpha-2-glycoprotein, an adipokine that leads to lipid degradation and higher insulin sensitivity in adipocytes (Garrido-Sánchez, PLoS One 2012, 7: e33264; Yang, Diabetes Care 2013, 36: 1074-82).

Circulating miR-122 as Novel Biomarker

The available epidemiological evidence on miR-122 and cardiometabolic conditions is sparse. Gao and colleagues (Gao, Lipids Health Dis 2012, 11: 55) reported higher miR-122 in patients with hyperlipidaemia, a positive correlation with total cholesterol, triglycerides, LDL-C, and presence of coronary artery disease. Surgery-induced weight loss resulted in marked reduction in miR-122 (Ortega, Clin Chem 2013, 59: 781-92). In the current study, we show—for the first time—that baseline levels of miR-122 are associated with development of metabolic syndrome and, more weakly, with T2DM. In contrast, there was no association of miR-122 with incident CVD, although the confidence interval of the risk ratio estimate was wide and a longer follow-up time might have been required to fully capture the long-term consequences of an adverse metabolic state on the cardiovascular system. Moreover, subgroup analysis unravelled a signal towards a positive association between miR-122 and CVD in individuals younger than 60 years. Notably, associations did not vary by degree of adiposity, a strong determinant of cardiometabolic risk (Würtz, PLoS Med 2014, 11: e1001765; Fall, PLoS Med 2013, 10: e1001474).

Statin treatment decreased both lipoprotein and miR-122 release from the liver. Since miR-122 is either absent from lipoproteins, including VLDL and HDL (Table 3), or only present at very low levels, i.e. in LDL (Vickers, Nat Cell Biol 2011, 13: 423-33), the pronounced effect of statins on circulating miR-122 levels cannot be explained by effects on plasma lipoproteins. Instead, it is most likely caused by reduced secretion of liver exosomes (FIG. 5), in which miR-122 has been localized in abundance (Huang, BMC Genomics 2013, 14: 319; Gallo, PLoS One 2012, 7: e30679). Circulating miR-122 is undetectable in exosome-depleted serum (Gallo, PLoS One 2012, 7: e30679). By inhibiting cholesterol synthesis, statins also modulate protein prenylation (Wang, Dev Cell 2008, 15: 261-71). This posttranslational modification promotes the membrane localisation of proteins, in particular of Rab27 proteins that control the different steps of exosome secretion (Jaé, FEBS Lett 2015, 589: 3182-8; Ostrowski, Nat Cell Biol 2010, 12: 19-30). Statins may reduce circulating miR-122 levels by inhibiting the prenylation of Rab proteins and hepatic exosome secretion. The latter might constitute a novel part of the beneficial pleotropic effects of statin medication.

Translational Potential of miR-122 in Clinical Practice

In addition to its metabolic effects, miR-122 has been associated with various liver diseases, including non-alcoholic fatty liver disease, non-alcoholic steatohepatitis, and viral hepatitis, and liver disease (Laterza, Biomark Med 2013, 7: 205-10; Zhang, Clin Chem 2010, 56: 1830-38). MiR-122 facilitates the replication, translation, and packaging of the hepatitis C virus, and thereby promotes a chronic infection with the virus (Lanford, Science 2010, 327: 198-201; Rottiers, Nat Rev Mol Cell Biol 2012, 13: 239-50). For this specific condition, antisense-based drugs targeting miR-122 are in development (Baek, Arch Pharm Res 2014, 37: 299-305; Lindow, J Cell Biol 2012, 199: 407-12). In a recent Phase 2a trial in 36 patients with chronic hepatitis C genotype 1 infection, treatment with the antisense oligonucleotide ‘miravirsen’ reduced hepatitis C virus RNA levels in a dose-dependent manner (Janssen, N Engl J Med 2013, 368: 1685-94). Two other ongoing Phase 2 clinical trials included patients who did not respond to treatment with pegylated-interferon α and ribavirin (NCT01872936, NCT02031133). The availability of therapeutic inhibitors of miR-122 provides an opportunity to test the effects of miR-122 intervention on cardiometabolic intermediate traits and diseases in patients (either as a secondary endpoint in ongoing trials, or as a primary endpoint).

Strengths and Limitations

Our study had several strengths and limitations. The prospective Bruneck cohort is extremely well-characterized with a 100% follow-up and high-quality ascertainment of clinical endpoints and potential confounders. To help identify independent associations, we adjusted for a large panel of potential confounding variables, including smoking, social class, physical activity, and adiposity measures. We incorporated repeat measurements of miR-122 in our statistical models, which is particularly important given that the within-person variability of miR-122 was high (correlation coefficient over 5 years: 0.24). In contrast to platelet-related miRNAs, which are reduced in diabetic patients (Willeit, Circ Res 2013, 112: 595-600; Elgheznawy, Circ Res 2015; 117: 157-65), miR-122 levels showed positive associations with metabolic syndrome and T2DM and were highly correlated in serum and plasma. Potential limitations of our study are that the Bruneck Study population was entirely Caucasian. The ASCOT trial involved hypertensive individuals at moderately elevated CVD risk, a somewhat select population. Finally, miR-122 was measured in serum and plasma. Expression data in the liver would be a more direct measure, but, clearly, this is not feasible in population studies.

### CONCLUSIONS

High circulating miR-122 levels are correlated with complex lipids containing saturated and monounsaturated fatty acids that can be derived from hepatic de novo lipogenesis and an adverse metabolic profile, inhibition of HMG-CoA reductase by atorvastatin reduces miR-122 release, and circulating miR-122 levels are associated with future development of metabolic syndrome and T2DM in the general population.

TABLE 2
Primer sequences used to quantify expression of genes implicated
in cholesterol and lipid metabolism at the mRNA level in
antagomiR-122 treated mice, related to FIG 1.

| Enzyme | Forward (5′→3′) | Reverse (3′→5′) |
|---|---|---|
| MTTP | AGGCAATTCGAGACAAAG | ACGTCAAAGCATATCGTTC |
| Acly | ATGCGAGTGCAGATCC | AAGGTAGTGCCCAATG |
| Cpt2 | ATGCTGTTCACGATGAC | CTCATTACCTTCAGTTGGG |
| ALDO | TGAATAGGCTGCGTTCTCTTG | GCAGTGCTTTCCTTTCCTAACTC |
| LDLR | GGTACTGGCAACCACCATTGGG | GCCAATCGACTCACGGGTTCAG |
| HMGCOA | CTTGTGGAATGCCTTGTGATTG | AGCCGAAGCAGCACATGAT |
| SREBP2 | GCGTTCTGGAGACCATGGA | ACAAAGTTGCTCTGAAAACAAATCA |
| SREBP1 | GCAGCCACCATCTAGCCTG | CAGCAGTGAGTCTCTGCCTTGAT |
| FASN | CTTCGCCAACTCTACCATGG | TTCCACACCCATGAGCGAGT |
| ACC1 | ATCCAGGCCATGTTGAGACG | AGATGTGCTGGGTCATGTGG |
| SCD1 | CTGGAGATCTCTGGAGCATGTGGG | TACCCTTTGCTGGCAGCCGA |
| CPT | TTGATCAAGAAGTGCCGGACGAGT | GTCCATCATGGCCAGCACAAAGTT |
| AMPK | TGACCGGACATAAAGTGGCTGTGA | TGATGATGTGAGGGTGCCTGAACA |
| GAPDH | ACACATTGGGGGTAGGAACA | AACTTTGGCATTGTGGAAGG |
| 18S | TTCCGATAACGAACGAGACTCT | TGGCTGAACGCCACTTGTC |

TABLE 3
MiRNAs in human HDL and VLDL. MiRNAs were measured using
RNA extracted from human HDL and VLDL samples.

Cₜ

| miRNA | VLDL | HDL |
|---|---|---|
| miR-24 | 28.8 | 32.0 |
| miR-92a | 31.8 | 35.8 |
| miR-122 | Not detectable | Not detectable |
| miR-126 | 30.0 | 29.1 |
| miR-146a | 30.9 | 36.1 |
| miR-191 | 30.0 | 33.9 |
| miR-223 | 23.5 | 24.8 |

Cₜ, Cycle threshold.

The present invention refers to the following nucleotide and amino acid sequences:

SEQ ID NO: 1: Nucleotide sequence of mature human miR-122

5′ uggagugugacaaugguguuug 3′

SEQ ID NO: 2: Nucleotide sequence of the seed sequence human miR-122

5′ ggagugu 3′

1. Method for identifying a subject which has a risk for developing metabolic syndrome and/or type-2 diabetes before onset of the disease(s), wherein the method comprises:
(a) analyzing in a sample obtained from a test subject the amount of miR-122; and
(b) identifying a subject, which has a risk for developing metabolic syndrome and/or type-2 diabetes, wherein an increased amount of miR-122 as compared to the amount of miR-122 of a healthy reference population indicates a risk for developing metabolic syndrome and/or type-2 diabetes.

2. MiR-122 as a predictive biomarker for use in identifying a risk for developing metabolic syndrome and/or type-2 diabetes before onset of the disease(s), wherein an increased amount of miR-122 as compared to the amount of miR-122 of a healthy reference population indicates a risk for developing metabolic syndrome and/or type-2 diabetes.

3. Method of claim 1, wherein the risk for developing a metabolic syndrome and/or type-2 diabetes is identified at least 1 year before clinical manifestation of the metabolic syndrome and/or the type-2 diabetes, respectively.

4. Method of claim 1 or biomarker for the use according to claim 2 or 3, wherein the method or the biomarker is for identifying a subject which has a risk for developing metabolic syndrome, and wherein the risk ratio is 1.8-4.6.

5. Method of claim 1, wherein the method or the biomarker is for identifying a subject which has a risk for developing type-2 diabetes, and wherein the risk ratio is 1.3-6.4.

6. Method of claim 1, wherein miR-122 is a polynucleotide selected from the following polynucleotides
(i) a polynucleotide comprising or consisting of the nucleotide sequence of SEQ ID NO: 1;
(ii) a polynucleotide which is at least 95% identical to the nucleotide sequence of SEQ ID NO: 1 and being functional, wherein the function comprises the activity to repress translation of the target genes of miR-122; and
(iii) a polynucleotide according to (ii), which comprises the nucleotide sequence of SEQ ID NO: 2.

7. Method of claim 1, wherein the method or the biomarker is for identifying a subject which has a risk for developing metabolic syndrome, and wherein said healthy reference population does not have metabolic syndrome and fulfills at least one of the criteria (i) and (ii), below:
(i) does not have any of the features selected from
(a) waist circumference of men that is ≥102 cm, or of women that is ≥88 cm,
(b) fasting triglycerides ≥50 mg/dl, or being on drug treatment for elevated triglycerides;
(c) HDL cholesterol in men that is <40, and in women that is <50 mg/dl or being on drug treatment for reduced HDL cholesterol;
(d) blood pressure ≥130/≥85 mmHg, or being on antihypertensive drug treatment and having a history of hypertension; and
(e) fasting glucose ≥100 mg/dl, or being on drug treatment for elevated glucose; or
(ii) does have a miR-122 level beneath the 33th percentile of population normative values.

8. Method of claim 1, wherein the method or the biomarker is for identifying a subject which has a risk for developing type-2 diabetes, and wherein said healthy reference population does not have type-2 diabetes and does have a miR-122 level beneath the 33th percentile of population normative values.

9. Method of claim 1, wherein an amount of miR-122 of the test subject, which is at least 110% of the amount of miR-122 of the healthy reference population, indicates the risk for developing metabolic syndrome and/or type-2 diabetes.

10. Method of claim 1, further comprising identifying whether said test subject has at least one of the risk factors selected from overweight, obesity, central obesity, hypertension, low HDL cholesterol or high triglyceride levels.

11. Method of claim 1, wherein a medication for a metabolic syndrome and/or type-2 diabetes is to be administered to the test subject which has been identified as having a risk for developing metabolic syndrome and/or type-2 diabetes; and/or
wherein life-style interventions are recommended to the test subject which has been identified as having a risk for developing metabolic syndrome and/or type-2.

12. Method of claim 1, wherein said sample is blood, blood plasma, blood serum, urine or a liver tissue sample.

13. Method of claim 1, wherein said amount of miR-122 is analyzed by quantitative PCR.

14. Method for monitoring the therapeutic success during the treatment of metabolic syndrome and/or type-2 diabetes, wherein the method comprises:
(a) analyzing in a first sample obtained from a test subject the amount of miR-122, wherein said first sample was obtained before or under treatment of the test subject with medication for metabolic syndrome and/or type-2 diabetes;
(b) analyzing in a second sample of said test subject the amount of miR-122, wherein said second sample was obtained under or after treatment of the test subject with medication for metabolic syndrome and/or type-2 diabetes; and
(c) predicting the therapeutic success, wherein a decreased amount of miR-122 in the second sample as compared to the first sample indicates therapeutic success.

15. Monitoring method of claim 14, wherein the method is for monitoring the therapeutic success during the treatment of type-2 diabetes.


SEQUENCE LISTING (36 sequences)

SEQ ID NO: 1 - RNA, 22 nt, Homo sapiens
  mature human miR-122
uggagugugacaaugguguuug

SEQ ID NO: 2-4 - skipped (3 entries)

SEQ ID NO: 5 - DNA, 21 bp, Artificial Sequence
  DNA oligonucleotides complementary to mature miR-122
aaacaccattgtcacactcca

SEQ ID NO: 6 - DNA, 41 bp, Artificial Sequence
  5S rRNA
caggcccgaccctgcttagcttccgagagatcagacgagat

SEQ ID NO: 7 - DNA, 18 bp, Artificial Sequence
  Primer MTTP
aggcaattcgagacaaag

SEQ ID NO: 8 - DNA, 19 bp, Artificial Sequence
  Primer MTTP reverse
acgtcaaagcatatcgttc

SEQ ID NO: 9 - DNA, 16 bp, Artificial Sequence
  Primer Acly
atgcgagtgcagatcc

SEQ ID NO: 10 - DNA, 16 bp, Artificial Sequence
  Primer Acly reverse
aaggtagtgcccaatg

SEQ ID NO: 11 - DNA, 17 bp, Artificial Sequence
  Primer Cpt2
atgctgttcacgatgac

SEQ ID NO: 12 - DNA, 19 bp, Artificial Sequence
  Primer Cpt2 reverse
ctcattaccttcagttggg

SEQ ID NO: 13 - DNA, 21 bp, Artificial Sequence
  Primer ALDO
tgaataggctgcgttctcttg

SEQ ID NO: 14 - DNA, 23 bp, Artificial Sequence
  Primer ALDO reverse
gcagtgctttcctttcctaactc

SEQ ID NO: 15 - DNA, 22 bp, Artificial Sequence
  Primer LDLR
ggtactggcaaccaccattggg

SEQ ID NO: 16 - DNA, 22 bp, Artificial Sequence
  Primer LDLR reverse
gccaatcgactcacgggttcag

SEQ ID NO: 17 - DNA, 22 bp, Artificial Sequence
  Primer HMGCOA
cttgtggaatgccttgtgattg

SEQ ID NO: 18 - DNA, 19 bp, Artificial Sequence
  Primer HMGCOA reverse
agccgaagcagcacatgat

SEQ ID NO: 19 - DNA, 19 bp, Artificial Sequence
  Primer SREBP2
gcgttctggagaccatgga

SEQ ID NO: 20 - DNA, 25 bp, Artificial Sequence
  Primer SREBP2 reverse
acaaagttgctctgaaaacaaatca

SEQ ID NO: 21 - DNA, 19 bp, Artificial Sequence
  Primer SREBP1
gcagccaccatctagcctg

SEQ ID NO: 22 - DNA, 23 bp, Artificial Sequence
  Primer SREBP1 reverse
cagcagtgagtctctgccttgat

SEQ ID NO: 23 - DNA, 20 bp, Artificial Sequence
  Primer FASN
cttcgccaactctaccatgg

SEQ ID NO: 24 - DNA, 20 bp, Artificial Sequence
  Primer FASN reverse
ttccacacccatgagcgagt

SEQ ID NO: 25 - DNA, 20 bp, Artificial Sequence
  Primer ACC1
atccaggccatgttgagacg

SEQ ID NO: 26 - DNA, 20 bp, Artificial Sequence
  Primer ACC1 reverse
agatgtgctgggtcatgtgg

SEQ ID NO: 27 - DNA, 24 bp, Artificial Sequence
  Primer SCD1
ctggagatctctggagcatgtggg

SEQ ID NO: 28 - DNA, 20 bp, Artificial Sequence
  Primer SCD1 reverse
taccctttgctggcagccga

SEQ ID NO: 29 - DNA, 24 bp, Artificial Sequence
  Primer CPT1
ttgatcaagaagtgccggacgagt

SEQ ID NO: 30 - DNA, 24 bp, Artificial Sequence
  Primer CPT1 reverse
gtccatcatggccagcacaaagtt

SEQ ID NO: 31 - DNA, 24 bp, Artificial Sequence
  Primer AMPK
tgaccggacataaagtggctgtga

SEQ ID NO: 32 - DNA, 24 bp, Artificial Sequence
  Primer AMPK reverse
tgatgatgtgagggtgcctgaaca

SEQ ID NO: 33 - DNA, 20 bp, Artificial Sequence
  Primer GAPDH
acacattgggggtaggaaca

SEQ ID NO: 34 - DNA, 20 bp, Artificial Sequence
  Primer GAPDH reverse
aactttggcattgtggaagg

SEQ ID NO: 35 - DNA, 22 bp, Artificial Sequence
  Primer 18S
ttccgataacgaacgagactct

SEQ ID NO: 36 - DNA, 19 bp, Artificial Sequence
  Primer 18S reverse
tggctgaacgccacttgtc